(19) United States
(12) Patent Application Publication
(10) Pub. No.: US 2025/0352787 A1
(43) Pub. Date: Nov. 20, 2025

(54) DELIVERY AND RETRIEVAL SYSTEM FOR MEDICAL DEVICE

(71) Applicant: Medtronic, Inc., Minneapolis, MN (US)

(72) Inventors: Vladimir Grubac, Brooklyn Park, MN (US); Ryan Butz, Oak Ridge, TN (US); Lydia C. Kirsch, St. Louis Park, MN (US); Allison E. Winans, Edina, MN (US)

(21) Appl. No.: 18/871,419

(22) PCT Filed: May 25, 2023
(86) PCT No.: PCT/US2023/067476

§ 371 Date: Dec. 3, 2024

Related U.S. Application Data
(60) Provisional application No. 63/366,368, filed on Jun. 14, 2022.

(51) Int. Cl. A61N 1/05; A61B 17/00

(52) U.S. Cl. CPC A61N 1/0573; A61B 17/00234; A61B 2017/00243; A61B 2017/00358; A61N 2001/0578

(57) ABSTRACT

A medical system configured to impart a torque to a medical device within a patient. The medical system includes a driver including a driver body supporting a torque coupler. A snare is configured to engage the medical device. The torque coupler is configured to receive the medical device within a coupler volume. The snare and/or the torque coupler are configured to exert forces on the medical device such that the torque coupler imparts a torque to a retrieval structure of the medical device, causing rotation of the medical device. In examples, the medical device includes an attachment member configured to engage or disengage tissue when the medical device rotates. The medical system may include a delivery catheter configured to deliver and/or retrieve the torque coupler, snare, and/or the medical device.

This application claims priority to U.S. Provisional Patent Application No. 63/366,368, filed 14 Jun. 2022, the entire contents of each of which are incorporated by reference herein.

### FIELD

This disclosure is related to system for delivery and/or retrieval of implantable medical devices.

### BACKGROUND

Various types of implantable medical devices have been implanted for treating or monitoring one or more conditions of a patient. Such implantable medical devices may be adapted to allow medical devices to monitor and/or treat conditions or functions relating to heart, muscle, nerve, brain, stomach, endocrine organs or other organs and their related functions. The implantable medical devices may be implanted at target locations selected to detect a physiological condition of the patient and/or deliver one or more therapies. For example, implantable medical devices may be delivered to locations within an atrium or ventricle of a heart to sense intrinsic cardiac signals and deliver pacing or antitachyarrhythmia shock therapy.

Some implantable medical devices are sized to be completely implanted within one of the chambers of the heart and/or another anatomical volume of the patient to detect a physiological condition and/or deliver one or more therapies. Such implantable medical devices may utilize delivery and/or retrieval systems to allow a clinician to navigate the implantable medical device (e.g., through vasculature of the patient) to the target location, and/or to retrieve the implantable medical device from the patient. In some examples, the implantable medical device may include one or more anchoring components intended to engage tissues at the target location (e.g., for implantation) and/or disengage from tissue at the target location (e.g., for retrieval).

### SUMMARY

The disclosure describes a medical system configured to deliver, position, retrieve, and/or otherwise re-orient an implantable medical device within an anatomical volume (e.g., a chamber of a heart) within a patient. The medical system includes a snare configured to engage the implantable medical device and a torque coupler configured to receive at least some portion of the implantable medical device. The snare is configured to exert a proximally-directed force on the IMD. The torque coupler is configured to exert a distally-directed force on a retrieval structure of the IMD when the torque coupler receives the IMD. The snare and the torque coupler are configured such that the proximally directed force and/or the distally-directed force generate a contact force from the torque coupler to the IMD retrieval structure. The medical system is configured to transfer a torque from the torque coupler to the IMD when the contact force provides an engagement force. In examples, the medical system includes a delivery catheter configured to deliver and/or retrieve the torque coupler, the intermediate member, and/or the implantable medical device through vasculature of the patient.

In an example, a medical system, comprises: a driver including a driver body and a torque coupler configured to receive a torque from the driver body, wherein the driver body defines a lumen which opens to a coupler volume defined by the torque coupler, wherein the torque coupler defines a coupler axis extending through the coupler volume and a plurality of protrusions extending toward the coupler axis, wherein the coupler volume is configured to receive a retrieval structure of an implantable medical device when the coupler axis defines an oblique angle with an IMD axis of the implantable medical device, and wherein the torque coupler is configured to exert a distally-directed force on the retrieval structure when the coupler volume receives the retrieval structure; and a snare configured to translate within the lumen, wherein the snare is configured to exert a proximally-directed force on the implantable medical device when the snare translates within the lumen, wherein the snare and the torque coupler are configured to generate an engagement force from the torque coupler to the retrieval structure when at least one of the snare exerts the proximally-directed force or the torque coupler exerts the distally-directed force, wherein the plurality of protrusions are configured to transfer the torque from the torque coupler to the retrieval structure when: the coupler volume receives the retrieval structure, the coupler axis defines the oblique angle with the IMD axis, the snare and the torque coupler generate the engagement force, and at least a first protrusion of the plurality of protrusions contacts the retrieval structure.

In an example, a technique comprises: imparting, by a driver body of a driver, a torque on a torque coupler of the driver, wherein the driver body defines a lumen opening to a coupler volume defined by the torque coupler, wherein the torque coupler defines a coupler axis extending through the coupler volume and a plurality of protrusions extending toward the coupler axis, and wherein the coupler volume is configured to receive a retrieval structure of an implantable medical device when the coupler axis defines an oblique angle with an IMD axis of the implantable medical device; imparting at least one of a distally-directed force, exerted by the torque coupler, on the retrieval structure or a proximally-directed force, exerted by a snare, on the IMD to generate an engagement force from the torque coupler to the retrieval structure, wherein the snare is configured to translate within the lumen; and transferring the torque from the torque coupler to the retrieval structure using the plurality of protrusions when the coupler volume receives the retrieval structure, the coupler axis defines the oblique angle with the IMD axis, and at least a first protrusion of the plurality of protrusions contacts the retrieval structure.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a conceptual drawing illustrating an example medical system and delivery catheter within a heart.

FIG. 2 is a schematic diagram illustrating an example medical system including a snare, a torque coupler, and a delivery catheter.

FIG. 3 is a cross-sectional diagram of the example medical system of FIG. 2.

FIG. 4 is a schematic diagram of the example medical system with the snare distal to the torque coupler.

FIG. 5 is a schematic diagram of the example medical system with the snare engaging an implantable medical device.

FIG. 6 is a schematic diagram of the example medical system with a snare sheath distal to the torque coupler.

FIG. 7 is a schematic diagram of the example medical system with the torque coupler engaging the implantable medical device at a first oblique angle.

FIG. 8 is a schematic diagram of the example medical system with the torque coupler engaging the implantable medical device at second oblique angle.

FIG. 9 is a schematic diagram of the example medical system within a delivery receptacle of a delivery catheter.

FIG. 10 is a perspective diagram of an example torque coupler and an implantable medical device.

FIG. 11A is a top plan view of an example torque coupler.

FIG. 11B is a cross-sectional diagram of the example torque coupler of FIG. 11B.

FIG. 12 is a perspective diagram of an example torque coupler with a snare engaging an implantable medical device.

FIG. 13 is a schematic diagram of an example torque coupler defining a non-planar coupler perimeter.

FIG. 14 is a perspective diagram of an example torque coupler and a snare engaging an implantable medical device.

FIG. 15 is a cross-sectional diagram of an example torque coupler and a snare engaging an implantable medical device.

FIG. 16 illustrates an example technique for transferring a torque to an implantable medical device.

### DETAILED DESCRIPTION

This disclosure describes a medical system configured to deliver, position, and/or retrieve an implantable medical device (“IMD”) within an anatomical volume (e.g., a chamber of a heart) within a patient. The medical system is configured to receive a torque (e.g., from a clinician) and impart the torque to the IMD to implant, retrieve, re-position, and/or re-orient the IMD in the anatomical volume. In examples, the medical system is configured to enable a rotation of the IMD around an IMD axis when the medical system imparts the torque. The rotation of the implantable medical device may cause an attachment member of the implantable medical device to engage tissues at a target site, disengage tissues at the target site, and/or otherwise cause a re-positioning and/or re-orientation of the medical device within the patient.

The medical system includes a driver (e.g., an elongate body) configured to receive the torque (e.g., from the clinician) and impart the torque to a torque coupler at a distal end of the driver. In examples, the torque coupler is a receptacle or a cup, such as a torque cup or torque receptacle. The torque coupler defines a coupler volume configured to receive at least some portion of the implantable medical device. In examples, the driver is configured to position at least partially within vasculature of a patient when the torque coupler is positioned within the anatomical volume. In some examples, the driver includes a proximal portion configured to be extracorporeal to a patient and a distal portion configured to be intracorporeal to the patient. The distal portion may support the torque coupler. The medical system may be configured such that a torque imparted to the proximal portion (e.g., by a clinician) causes the distal portion to impart the torque to the torque coupler. The medical system is configured to transfer the torque from the torque coupler to the IMD to implant, retrieve, re-position, and/or re-orient the IMD. In examples, the IMD includes an attachment member (e.g., a helix) configured to engage tissue or disengage from tissue based on a rotation of the IMD. The medical system may transfer the torque from the torque coupler to the IMD to cause the IMD rotation, such that the attachment member engages with or disengages from the tissue.

The torque coupler includes a surface (“coupler surface”) defining the coupler volume and an opening (“coupler opening”) to the coupler volume at a distal end of the torque coupler (“coupler distal end”). In examples, the torque coupler defines an axis (“coupler axis”) extending through the coupler volume and the coupler opening. The coupler opening is configured to allow at least a portion of a IMD retrieval structure of the IMD (“IMD retrieval structure”) to pass therethrough when the coupler volume receives the implantable medical device. The torque coupler (e.g., the coupler surface) defines a plurality of protrusions extending toward the coupler axis. The plurality of protrusions are configured to engage the IMD retrieval structure to transfer torque to the IMD when the coupler volume receives the IMD retrieval structure and a torque is exerted (e.g., by a clinician) on the driver.

As used herein, when the torque coupler (e.g., the coupler volume) receives the IMD retrieval structure, this means the torque coupler receives at least a portion of the IMD retrieval structure. For example, the torque coupler may receive a distal portion of the IMD retrieval structure, some other portion of the IMD retrieval structure, or substantially the entirety of the IMD retrieval structure. The IMD retrieval structure may be any structure and/or feature of the IMD configured to receive a torque from the torque coupler when the torque coupler (e.g., the coupler volume) receives the IMD.

The torque coupler is configured to receive the IMD retrieval structure over a range of relative orientations between the coupler axis and an IMD axis defined by the IMD and extending through the IMD retrieval structure. The torque coupler may be configured to receive the IMD retrieval structure when the coupler axis and the IMD axis are offset, such that the coupler axis and the IMD axis define an oblique angle (e.g., an oblique angle of about 30 degrees). The protrusions may be configured to engage the IMD retrieval structure when the torque coupler receives the IMD retrieval structure (e.g., at an oblique angle) and torque coupler rotates relative to the IMD retrieval structure. In examples, the IMD retrieval structure defines a plurality of petals extending in a direction substantially radial to the IMD axis, and the protrusions are configured to engage one or more of the petals when torque coupler rotates relative to the IMD retrieval structure (e.g., at an oblique angle).

The use of a torque coupler configured to receive the IMD at a substantially oblique angle to the IMD may ease mechanical coupling requirements that might otherwise be present when using a cup to capture and/or torque the IMD. For example, in some systems a cup or receptacle may be configured as a socket intended to mechanically mate with a proximal end of an IMD to transfer a torque. The cup may define recesses configured to receive protrusions defined by the IMD, and/or define protrusions configured to insert into recesses defined by the IMD. Transfer of a torque from the cup to the IMD may thus require relatively precise alignment (e.g., a substantially parallel and/or coincident alignment) between an axis of the cup and an IMD axis in order to achieve the necessary mechanical mating. Achieving this relatively precise alignment may present difficulty when the cup and the device are positioned within a constrained anatomical space of a patient (e.g., a chamber of a heart). In contrast, use of a torque coupler configured to receive and/or engage the IMD at a substantially oblique angle to the IMD may alleviate this alignment burden.

The medical system includes a snare defining a body (“snare body”) and a loop (“snare loop”) at a distal end of the snare body. The snare (e.g., the snare loop”) is configured to engage the IMD (e.g. the IMD retrieval structure). The snare may be configured to engage the IMD to, for example, guide the torque coupler toward the IMD retrieval structure and/or cause the torque coupler to receive the IMD retrieval structure. In examples, a body of the driver (“driver body”) defines a lumen (“driver lumen”) and a lumen opening (“driver lumen opening”) which opens into the coupler volume. The snare may be configured to slidably translate within the driver lumen, such that a clinician may cause the snare to translate proximally and/or distally within the driver lumen, and/or cause the driver and the torque coupler to translate relative to the snare. For example, the snare may be configured to extend through the driver lumen and distal to the torque coupler such that the snare loop may engage the IMD (e.g., the IMD retrieval structure) when the IMD is distal to the torque coupler. A clinician may cause the driver and the torque coupler to translate distally over the snare when the snare loop is engaged with the IMD, such that the snare substantially guides the torque coupler toward the IMD retrieval structure.

The snare is configured to exert a proximally-directed force on the IMD (e.g., a force acting on the IMD in a direction toward the torque coupler) when the snare engages the IMD. The torque coupler is configured to exert a distally-directed force on the IMD retrieval structure (e.g., a force acting on the IMD in a direction toward the IMD) when the torque coupler receives the IMD retrieval structure. For example, a clinician may push the torque coupler (using the driver body) against the IMD retrieval structure to cause the torque coupler to exert the distally-directed force on the IMD retrieval structure. The clinician may pull the IMD (using the snare) toward the torque coupler to cause the snare to exert the proximally-directed force on the IMD. The medical system is configured such that, when the torque coupler is in contact with the IMD retrieval structure (e.g., when the torque coupler receives the IMD retrieval structure), the proximally-directed force and/or the distally directed force generate a contact force exerted from the torque coupler to the IMD retrieval structure.

As used herein, a contact force exerted from the torque coupler to the IMD retrieval structure may include one or more action forces and/or one or more reaction forces. For example, the contact force may include an action force imparted by torque coupler to the IMD retrieval structure when the torque coupler exerts the distally-directed force. The contact force may include a reaction force generated by the torque coupler in response to the snare exerting the proximally-directed force on the IMD and the IMD transmitting the proximally directed force to the torque coupler. Hence, the medical system is configured such that a clinician may control the contact force exerted from the torque coupler to the IMD retrieval structure using the driver and the snare in combination or individually.

The clinician may control the contact force to improve and/or control torque transfer when contact is established between the torque coupler and the IMD retrieval structure. For example, the torque coupler may approach the IMD along a path substantially offset (e.g., oblique to) an IMD axis of the IMD, such that when the torque coupler receives the IMD retrieval structure, the coupler axis and the IMD axis define an oblique angle. The clinician may cause the torque coupler to rotate about the coupler axis relative to the IMD retrieval structure (e.g., as the coupler axis and the IMD axis define the oblique angle). The contact force imparted from the torque coupler to the IMD retrieval structure may be increased (e.g., by the clinician) to cause one or more of the protrusions to sufficiently engage the IMD retrieval structure for the transfer of torque. Hence, the torque coupler may be configured to engage and/or transfer torque to an IMD when the torque coupler approaches the IMD along a path substantially offset (e.g., oblique to) an IMD axis of the IMD, easing any alignment requirements between the torque coupler and the IMD that might otherwise present difficulty within a constrained anatomical space of a patient (e.g., a chamber of a heart).

In examples, the contact force may be varied (e.g., by the clinician) such that the contact force is either an engagement force or a slipping force. An engagement force is a contact force having a force magnitude sufficient to cause at least a one protrusion to transfer torque from the torque coupler to the IMD retrieval structure when the protrusion contacts the IMD retrieval structure. The slipping force is a contact force having a force magnitude insufficient to cause the at least one protrusion to transfer torque from the torque coupler to the IMD retrieval structure when the protrusion contacts the IMD retrieval structure. In examples, the plurality of protrusions are configured to slidably translate around (e.g., slip over) the IMD retrieval structure when the torque coupler rotates relative to the IMD retrieval structure, until the contact force is increased (e.g., by the clinician) from a slipping force to an engagement force. The plurality of protrusions may be configured such that, when the contact force increases to the engagement force, at least the one protrusion transfer a torque to the IMD sufficient to cause a rotation of the IMD about the IMD axis.

A force magnitude of the engagement force necessary to cause the first protrusion to transfer torque may be dependent on an orientation of the torque coupler with respect to the IMD (e.g., dependent on the oblique angle). For example, the engagement force may have a first engagement force magnitude when the torque coupler and the IMD define a first oblique angle and a second engagement force magnitude when the torque coupler and the IMD define a second oblique angle, wherein the first engagement force magnitude is different from (e.g., greater than or less than) the second engagement force magnitude. Likewise, a force magnitude of the slipping force may be dependent on an orientation of the torque coupler with respect to the IMD. The slipping force may have a first slipping force magnitude when the torque coupler and the IMD define the first oblique angle and a second slipping force magnitude when the torque coupler and the IMD define the second oblique angle, wherein the first slipping force magnitude is different from the second slipping force magnitude.

Hence, the medical system is configured such that the proximally-directed force exerted by the snare on the IMD and the distally-directed force exerted by the torque coupler on the IMD retrieval structure generate a variable contact force from the torque coupler to the IMD retrieval structure. The contact force may be an engagement force sufficient to cause the torque coupler to transfer a torque to the IMD. The contact force may be a slipping force insufficient to cause the torque coupler to transfer the torque to the IMD. The proximally-directed force and/or the distally directed force may be controlled by a clinician to provide an engagement force magnitude and/or a slipping force magnitude appropriate for the orientation of the torque coupler with respect to the IMD.

The plurality of protrusions may be configured to substantially slidably translate around (e.g., slip over) the IMD retrieval structure when the contact force is insufficient to cause the plurality of protrusions to transfer torque to the IMD retrieval structure (e.g., when the contact force provides a slipping force) and the torque coupler rotates relative to the IMD retrieval structure. The plurality of protrusions may be configured to slip over the IMD retrieval structure until at least one protrusion engages the IMD retrieval structure sufficiently to transfer the torque (e.g., until the contact force provide an engagement force). In examples, the IMD retrieval structure defines a plurality of IMD petals extending substantially radially from the IMD axis defined by the IMD. The plurality of protrusions may be configured to slidably translate around the plurality of IMD petals to until one or more protrusions engages a petal sufficiently to cause a rotation of the IMD about the IMD axis. In examples, the IMD retrieval structure defines at least a first IMD petal and a second IMD petal and a recess between the first IMD petal and the second IMD petal. The plurality of protrusions may be configured to slidably translate over the IMD retrieval structure through the recess until at least one protrusion inserts into the recess and engages the first petal and/or second petal sufficiently to cause the IMD to rotate about the IMD axis.

The plurality of protrusions may include at least a first protrusion and a second protrusion defined by the coupler surface of the torque coupler. The coupler surface further defines a base portion separating the first protrusion and the second protrusion. The first protrusion, second protrusion, and/or base portion may be configured to substantially slidably translate around the IMD retrieval structure when the contact force provide a slipping force. In examples, the first protrusion, second protrusion, and/or base portion define one or more rounded corners to assist the first protrusion, second protrusion, and/or base portion in slidably translating over the IMD retrieval structure. In examples, the plurality of IMD petals define one or more rounded corners. In some examples, at least one IMD petal includes a rounded lobe defining a curved, curvilinear, and/or polygonal curved profile in a geometric plane substantially parallel to, perpendicular to, or including the IMD axis of the IMD.

At least one protrusion (e.g., the first protrusion) may be configured to transmit a force to the IMD retrieval structure to transfer torque from the torque coupler to the IMD. In examples, the first protrusion may be configured to substantially distribute the force over a protrusion bearing surface when the plurality of protrusions transmit the torque to the IMD. In some examples, the protrusion bearing surface may be configured to transmit the force to an IMD bearing surface defined by an IMD petal. The IMD bearing surface may be configured to receive the force from the first protrusion as a distributed force over the IMD bearing surface. In examples, the IMD bearing surface extends substantially radially outward from an IMD axis of the IMD toward a distal end of the IMD petal. Hence, the IMD petal may be configured to receive a force from the first protrusion over an area extending radially outward toward the petal distal end rather than, for example, necessarily receiving the force as a substantially concentrated force generally in the area of the distal end of the IMD petal. This may assist in the transfer of torque from the torque coupler to the IMD when the torque coupler receives the IMD at a substantially oblique angle.

In examples, the medical system is configured to cause the torque coupler to reduce (and in some cases, even substantially eliminate) the oblique angle defined between the coupler axis and the IMD axis. Reducing the oblique angle may increase the effectiveness of torque transfer from the torque coupler to the IMD. For example, when the torque coupler contacts the IMD retrieval structure at an oblique angle, increasing the contact force may cause the torque coupler to pivot toward and/or slidably translate over the IMD retrieval structure to reduce the oblique angle Reducing the oblique angle may improve the contact between the protrusions of the torque coupler and IMD retrieval structure (e.g., the one or more petals of the IMD retrieval structure), and/or cause additional protrusions of the torque coupler to engage the IMD retrieval structure, such that the torque coupler may more effectively transfer torque to the IMD.

For example, the plurality of protrusions may be configured such that, when the first protrusion inserts into a first recess defined by the plurality of IMD petals, the second protrusion may insert into a second recess defined by the plurality of IMD petals. In examples, the medical system is configured such that, when the first protrusion is inserted in the first recess, decreasing an oblique angle between the torque coupler and the IMD (e.g., by increasing the contact force) causes the torque coupler to pivot toward the IMD retrieval structure, causing the second protrusion to insert into the second recess. Reducing and/or substantially eliminating the oblique angle may improve the contact between the protrusions of the torque coupler and IMD retrieval structure (e.g., the one or more petals of the IMD retrieval structure), and/or cause additional protrusions of the torque coupler to engage the IMD retrieval structure, such that the torque coupler may more effectively transfer torque to the IMD.

In examples, the medical system is configured to control (e.g., increase and/or decrease) the contact force between the torque coupler and the IMD while limiting an overall resultant force on the IMD. For example, the medical system may be configured such that the proximally-directed force exerted by the snare and the distally directed force exerted by the torque coupler provide counter-acting forces on the IMD. The counter-acting forces may increase and/or decrease the contact force while limiting the overall resultant force on the IMD. For example, increasing the proximally-directed force exerted by the snare while increasing the distally-directed force exerted by the torque coupler may increase the contact force while limiting the resultant force (e.g., the difference between the proximally-directed force and the distally-directed force) acting on the IMD. Decreasing the proximally-directed force exerted by the snare while decreasing the distally-directed force exerted by the torque coupler may decrease the contact force while limiting the resultant force acting on the IMD. Limiting the resultant force acting on the IMD may improve positional stability of the IMD within the patient while implanting, retrieving, re-positioning, and/or re-orienting the IMD.

The medical system may include a delivery catheter including a delivery receptacle or receptacle. The delivery receptacle may define a delivery receptacle volume configured to receive the torque coupler, the snare and at least a portion of the IMD. The delivery catheter may define a delivery catheter lumen and a delivery lumen opening which opens to the delivery receptacle volume. The driver (e.g., the driver body) may be configured to slidably translate within the delivery catheter lumen and through the delivery lumen opening, such that relative movement between the driver and the delivery catheter may cause the torque coupler, the snare, and/or at least the portion of the IMD to position within the delivery receptacle volume and/or exit the delivery receptacle volume (via the delivery lumen opening). The delivery catheter may be configured to transition through the vasculature of a patient, such that the torque coupler, the snare, and/or the IMD may be retrieved from and/or delivered to an anatomical volume of the patient (e.g., a heart chamber).

Hence, the medical system is configured to engage an IMD retrieval structure when a torque coupler of the medical system approaches the IMD along a path substantially offset (e.g., oblique to) an IMD axis of the IMD. The medical system is configured such that, when the torque coupler rotates relative to the IMD, at least a first protrusion of the torque coupler may engage the IMD retrieval structure to transfer a torque to the IMD. The medical system is configured such that a clinician may control a contact force (e.g., to provide an engagement force) from the torque coupler to the IMD retrieval structure to cause the first protrusion to engage the IMD retrieval structure. In examples, the clinician may control (e.g., increase) the contact force to further cause a second protrusion to engage the IMD retrieval structure. Hence, the medical system is configured to significantly ease the transfer of torque from the torque cap to the IMD within a constrained anatomical space of a patient (e.g., a chamber of a heart).

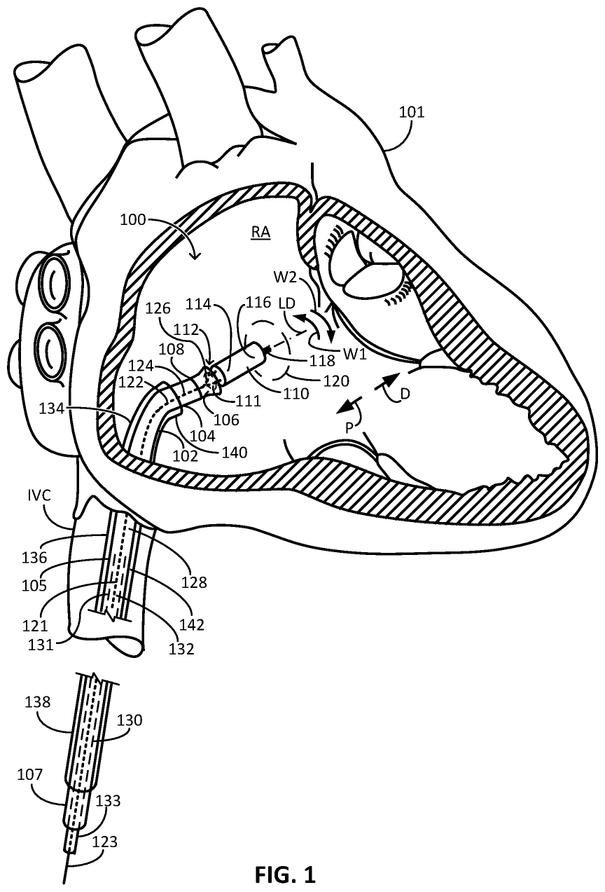

FIG. 1 is a conceptual diagram illustrating an example medical system 100 within a right atrium (“RA”) of a heart 101. Medical system 100 includes driver 102 including driver body 104. Driver body 104 may include a distal portion 105 (“driver body distal portion 105”) configured to be intracorporeal to the patient and a proximal portion 107 (“driver body proximal portion 107”) which may be extracorporeal to the patient when driver body distal portion 105 is intracorporeal. A torque coupler 106 is supported at a distal end 108 of driver body 104 (“driver body distal end 108”). In some examples, torque coupler 106 and driver body 104 may be substantially separate components. In some examples, torque coupler 106 may be substantially contiguous with driver body 104, such that torque coupler 106 and driver body 104 define a unified component.

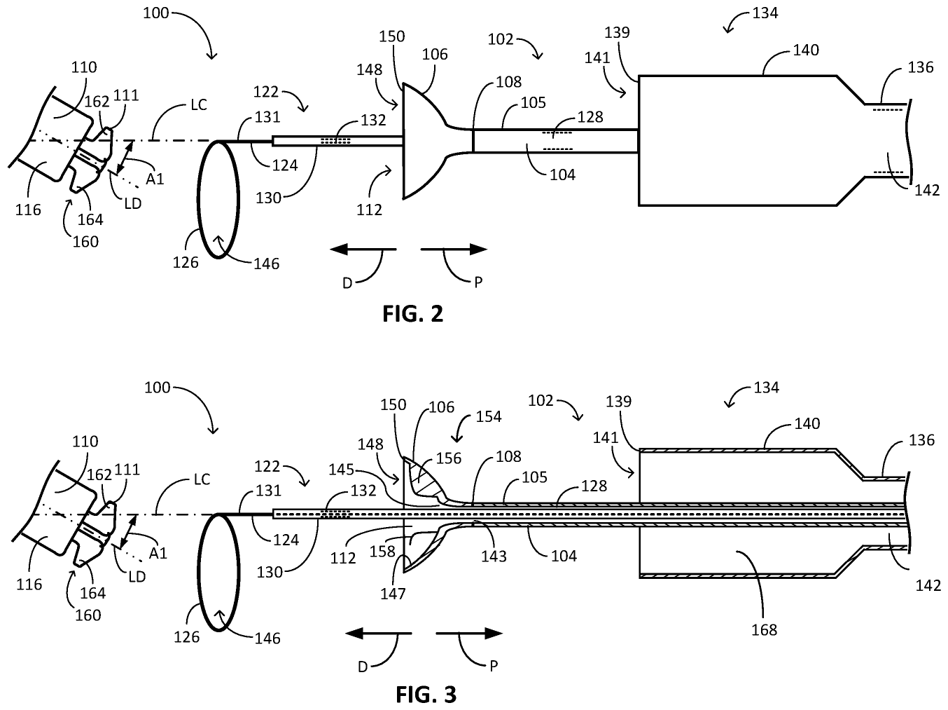

An IMD 110 is positioned within (e.g., inserted into) a coupler volume 112 defined by torque coupler 106. In the example of FIG. 1, a proximal portion 114 of IMD 110 (“IMD proximal portion 114”) including a retrieval structure 111 (“IMD retrieval structure 111”) is positioned within coupler volume 112. IMD retrieval structure 111 may be configured to engage with medical system 100 and/or another medical device to, for example, implant IMD 110 within an anatomical volume, retrieve IMD 110 from an anatomical volume, re-position IMD 110 within an anatomical volume, and/or re-orient IMD 110 within an anatomical volume. In FIG. 1, IMD proximal portion 114 (e.g., retrieval structure 111) is shown within coupler volume 112 and illustrated with dashed lines for clarity. IMD 110 may include a distal portion 116 (“IMD distal portion 116”) opposite IMD proximal portion 114. In examples, torque coupler 106 includes a coupler surface (e.g., coupler surface 147 (FIGS. 2, 3)) defining coupler volume 112.

In examples, IMD 110 (e.g., IMD distal portion 116) supports an attachment member 118 configured to engage tissue within a target site 120 of an anatomical volume. Attachment member 118 may be supported in IMD distal portion 116. In some examples, attachment member 118 is configured (e.g., as a helix) such that rotation of IMD 110 about an IMD axis LD defined by IMD 110 causes attachment member to engage and/or disengage tissues with target site 120. For example, attachment member 118 may be configured such that rotation of IMD 110 in a first rotational direction W1 about IMD axis LD causes attachment member 118 to engage (or alternately, disengage from) tissues within target site 120. Attachment member 118 may be configured such that rotation of IMD 110 about IMD axis LD in a second rotational direction W2 substantially opposite first rotational direction W1 causes attachment member 118 to disengage from (or alternately, engage) tissues within target site 120. In examples, IMD 110 includes one or more components (e.g., a communication antenna, a sensor, or another component) configured to rotate around and or revolve about IMD axis LD when IMD 110 rotates about IMD axis LD. Medical system 100 may cause IMD 110 to rotate about IMD axis LD to cause one or more of the components to substantially establish a specific orientation with respect to the anatomy of the patient, a another device implanted within or worn by the patient, another device external to the patient, and/or other devices.

Driver body 104 is configured to receive a torque (e.g., from a clinician) and transfer the torque to torque coupler 106. Torque coupler 106 (e.g., coupler surface 147) defines one or more protrusions (e.g., protrusions 154, FIG. 2-3) configured to engage IMD retrieval structure 111 to transfer torque to IMD 110 when coupler volume 112 receives IMD retrieval structure 111 and a torque is exerted (e.g., by a clinician) on driver 102 (e.g., on driver body proximal portion 107). Hence, medical system 100 is configured to mechanically couple torque coupler 106 and IMD 110 using the one or more protrusions, such that a torque imparted to driver body 104 (e.g., by a clinician) may cause rotation of IMD 110 in first rotational direction W1 and/or second rotation direction W2 to, for example, implant IMD 110, retrieve IMD 110, re-position IMD 110, and/or re-orient IMD 110 within an anatomical volume such as the RA.

Medical system includes a snare 122 defining a body 124 (“snare body 124”) and a loop 126 (“snare loop 126”) at a distal end of snare body 124. Snare loop 126 is configured to engage IMD 110 (e.g., IMD retrieval structure 111) to, for example, guide torque coupler 106 toward IMD retrieval structure 111, cause torque coupler 106 to receive IMD retrieval structure 111 within coupler volume 112, and/or control a contact force between torque coupler 106 and IMD 110 (e.g., IMD retrieval structure 111). In examples, driver body 104 defines a lumen 128 (“driver lumen 128”) and a lumen opening (e.g., driver lumen opening 143, FIG. 2-3) opening to coupler volume 112. Snare 122 may be configured to slidably translate within driver lumen 128, such that a clinician may cause snare 122 to translate proximally (e.g., in the direction P) and/or distally (e.g., in the direction D) within driver lumen 128, and/or cause driver 102 and torque coupler 106 to translate proximally and/or distally relative to snare 122. For example, snare 122 may be configured to extend through driver lumen 128 and distal to torque coupler 106 to engage IMD 110. A clinician may cause driver 102 and torque coupler 106 to translate distally over snare 122 (e.g., over snare body 124 and/or snare loop 126) when snare loop 126 is engaged with IMD 110, such that snare 122 substantially guides torque coupler 106 toward IMD retrieval structure 111.

Torque coupler 106 is configured such that coupler volume 112 may receive IMD retrieval structure 111 when torque coupler 106 (e.g., guided by snare 122) approaches IMD retrieval structure 111 along paths substantially offset (e.g., oblique to) IMD axis LD of IMD 110. Hence, torque coupler 106 may approach IMD retrieval structure 111 over a range of paths relative to IMD retrieval structure 111. The range of paths available eases alignment difficulties that might otherwise be present within a constrained anatomical space of a patient (e.g., the RA). For example, torque coupler 106 may be configured to receive IMD retrieval structure 111 when torque coupler 106 approaches IMD retrieval structure 111 along a path substantially offset from IMD axis LD by an oblique angle of about 30 degrees, about 20 degrees, or some other oblique angle. Further, medical system 100 is configured to such that a clinician may increase a contact force between torque coupler 106 and IMD 110 using snare 122 and/or driver body 104. For example, the clinician may cause snare 122 to exert a proximally-directed force on IMD retrieval structure 111 when snare loop 126 is engaged with IMD retrieval structure 111. The clinician may cause torque coupler 106 to exert a distally-directed force (e.g., via driver body 104) on IMD retrieval structure 111 when torque coupler 106 receives IMD retrieval structure 111. The increased contact force may cause one or more of protrusions of the torque coupler 106 to engage IMD retrieval structure 111 sufficiently to transfer torque to IMD retrieval structure 111. The increased contact force may cause torque coupler 106 to substantially pivot toward or otherwise re-orient with respect to IMD retrieval structure 111, reducing the oblique angle between torque coupler 106 (e.g., coupler axis LC) and IMD axis LD to, for example, improve the torque transfer from torque coupler 106 to IMD retrieval structure 111. Thus, medical system 100 may reduce the burden on a clinician when using medical system 100 to transfer torque to IMD 110 within a constrained anatomical space of a patient (e.g., the RA).

In some examples, snare 122 includes a sheath 130 (“snare sheath 130”) defining a lumen 132 (“sheath lumen 132”). Snare sheath 130 may be configured to slidably translate within driver lumen 128. In FIG. 1, snare sheath 130 is shown in dashed lines within driver lumen 128 for clarity. Snare body 124 and/or snare loop 126 may be configured to slidably translate within sheath lumen 132, such that a clinician may cause movement of snare sheath 130 (e.g., distal and/or proximal relative movement) relative to snare body 124 and/or snare loop 126. For example, a clinician may cause snare sheath 130 to translate distally relative to snare body 124 and snare loop 126 when snare loop 126 is engaged with IMD retrieval structure 111 to cause snare loop 126 to constrict around IMD retrieval structure 111. Snare sheath 130 may be configured to substantially guide torque coupler 106 toward IMD retrieval structure 111 subsequent to causing the constriction of snare loop 126 around IMD retrieval structure 111. In examples, snare body 124 includes a distal portion 121 (“snare body distal portion 121”) configured to be intracorporeal to the patient (e.g., via snare lumen 132 and/or driver lumen 128) and a proximal portion 123 (“snare body proximal portion 123”) which may be extracorporeal to the patient when snare body distal portion 121 is intracorporeal. Snare sheath 130 may include a distal portion 131 (“snare sheath distal portion 131”) configured to be intracorporeal to the patient (e.g., via driver lumen 128) and a proximal portion 133 (“snare sheath proximal portion 133”) which may be extracorporeal to the patient when snare sheath distal portion 131 is intracorporeal. Medical system 100 may be configured such that a clinician may cause relative movement between snare sheath 130 and snare body 124 and/or snare loop 126 using snare body proximal portion 123 and/or snare sheath proximal portion 133.

In some examples, medical system 100 includes a delivery catheter 134 configured to retrieve torque coupler 106, at least a portion of snare 122, and/or IMD 110 from an anatomical volume of the patient (e.g., the RA). In examples, delivery catheter 134 is configured to deliver torque coupler 106, at least a portion of snare 122, and/or IMD 110 to an anatomical volume of the patient. Delivery catheter 134 is illustrated as transparent in FIG. 1 for clarity. Delivery catheter 134 may include a distal portion 136 (“delivery catheter distal portion 136”) configured to be intracorporeal to the patient and a proximal portion 129 (“delivery catheter proximal portion 138”) which may be extracorporeal to the patient when delivery catheter distal portion 136 is intracorporeal. In examples, delivery catheter 134 is configured to deliver and/or retrieve torque coupler 106, at least a portion of snare 122, and/or IMD 110 using vasculature of a patient, such as an IVC or other vasculature leading to the anatomical volume.

In examples, delivery catheter 134 includes a delivery receptacle 140 or receptacle defining a delivery receptacle volume (e.g., delivery receptacle volume 168 (FIGS. 2-3)) configured to receive torque coupler 106, at least a portion of snare 122, and at least a portion of IMD 110. Delivery catheter 134 may define a lumen 142 (“delivery catheter lumen 142”) opening to delivery coupler volume 112. At least driver body 104 may be configured to slidably translate within delivery catheter lumen 142 and through the delivery lumen opening such that relative movement between driver body 104 and delivery catheter 134 may cause relative movement between torque coupler 106, at least a portion of snare 122, and/or IMD 110 and delivery catheter 134. Delivery receptacle 140 may define an opening (e.g., delivery receptacle opening 141 (FIGS. 2-3)) at a distal end of delivery receptacle 140 (e.g., delivery receptacle distal end 139, FIGS. 2-3). The delivery receptacle opening may be configured such that torque coupler 106, at least a portion of snare 122, and at least a portion of IMD 110 may pass therethrough.

Delivery catheter 134 may be configured to retrieve IMD 110 when IMD 110 is anchored to tissues within target site 120 (e.g., anchored by attachment member 118). For example, delivery catheter 134 may be configured to transition through vasculature of a patient to position torque coupler 106 and snare loop 126 in the proximity of IMD 110 when IMD 110 is anchored to tissues within target site 120. Delivery catheter 134 may be configured such that a force in the distal direction D on snare body 124 (e.g., by a clinician) causes snare loop 126 to extend distal to delivery receptacle 140 and/or torque coupler 106 (e.g., toward IMD 110) to engage IMD 110 (e.g., IMD retrieval structure 111). Delivery catheter 134 may be configured such that a force in the distal direction D on driver body 104 (e.g., exerted by a clinician) causes torque coupler 106 to extend distal to delivery receptacle 140 (e.g., guided by snare 122 engaged with IMD 110) such that torque coupler 106 engages IMD 110 (e.g., IMD retrieval structure 111). Delivery catheter 134 (e.g., delivery receptacle 140) may be configured to receive torque coupler 106, at least a portion of snare 122, and some portion of IMD 110 when attachment member 118 is disengaged (e.g., by torque transferred from torque coupler 106 to IMD 110) from target site 120. For example, delivery catheter 134 may move in the distal direction D relative to snare 122, torque coupler 106, and IMD 110 to receive snare 122, torque coupler 106, and at least the portion of IMD 110. Delivery catheter 134, torque coupler 106, snare 122, and IMD 110 may subsequently be withdrawn from the patient (e.g., via vasculature of the patient).

In examples, delivery catheter 134 is configured to position IMD 110 in proximity to target site 120 such that IMD 110 may be anchored to tissues within target site 120 (e.g., anchored by attachment member 118). For example, delivery catheter 134 may be configured to position torque coupler 106 and at least snare loop 126 within the delivery receptacle volume when IMD 110 is positioned within coupler volume 112 and snare loop 126 is engaged with IMD 110 (e.g., IMD retrieval structure 111). Delivery catheter 134 may be configured to traverse vasculature of the patient to position IMD 110 (e.g., attachment member 118) within or in proximity to target site 120. Medical system 100 may impart a torque to IMD 110 to cause attachment member 118 to engage tissues (e.g., tissue within target site 120) when attachment member 118 is within or in proximity to target site 120. Medical system 100 may be configured such that a force in the proximal direction P on driver body 104 (e.g., a proximal translation of driver body 104) causes torque coupler 106 to disengage from IMD 110 as IMD 110 remains anchored to tissues via attachment member 118. Medical system 100 may be configured such that snare 122 and/or snare sheath 130 may be translated to cause snare loop 126 to disengage from IMD 110 as IMD 110 remains anchored to tissues via attachment member 118. Delivery catheter 134, torque coupler 106, and snare 122 may subsequently be withdrawn from the patient (e.g., via vasculature of the patient).

Although the examples herein discuss delivery, retrieval, and/or positioning of IMD 110 within the RA of heart 101, medical system 100 may be configured to position IMD 110 in any of the other chambers of heart 101 and/or in other anatomical volumes of a patient in a like manner as that described for the RA of heart 101. Further, although the examples herein discuss attachment member 118 defining a helix, attachment member 118 may defines other structures, such as one or more elongated tines extending from, for example, IMD distal portion 116. Target site 120 may include an appendage of the RA, or the triangle of Koch region of the RA, or some other portion of heart 101, or some other location within a body of a patient.

FIG. 2 illustrates a schematic illustration of medical system 100 defining longitudinal axis L and including snare 122, driver 102, and delivery catheter 134. FIG. 3 illustrates medical system 100 with a cross section view of driver 102 and delivery catheter 134, with the cross-section cutting plane taken through longitudinal axis L. Medical system 100 is positioned relative to IMD 110 such that a coupler axis LC defined by torque coupler 106 defines an oblique angle A1 with device axis LD of IMD 110. As will be discussed, snare 122 may be configured to translate within driver lumen 128 both distally (e.g., in the distal direction D) and/or proximally (e.g., in the proximal direction P) relative to driver 102 and delivery catheter 134. Driver 102 may be configured to translate within delivery catheter lumen 142 both distally and/or proximally relative to snare 122 and delivery catheter 134. Delivery catheter 134 may be configured to translate both distally and/or proximally relative to snare 122 and driver 102. Driver body 104 defines a lumen opening 143 (“driver lumen opening 143”) configured to open into driver lumen 128. Snare 122 may extend through driver lumen opening 143 to translate within driver lumen 128.

Snare 122 is configured to engage IMD retrieval structure 111 when snare loop 126 is positioned around or in proximity to IMD retrieval structure 111. For example, snare loop 126 may define a loop aperture 146 configured to receive (e.g., substantially surround) at least some portion of IMD retrieval structure 111 when snare loop 126 is positioned around or in proximity to IMD retrieval structure 111. Snare 122 may be configured to cause snare loop 126 (e.g., loop aperture 146) to substantially constrict around IMD retrieval structure when snare loop 126 is engaged with IMD retrieval structure 111. For example, snare 122 may be configured such that, when snare loop 126 engages IMD retrieval structure 111, a proximal force (e.g., a force in the proximal direction P) exerted on snare body 124 causes snare loop 126 (e.g., loop aperture 146) to constrict around IMD retrieval structure 111.

In some examples, when at least snare body 124 is within sheath lumen 132, snare sheath 130 may translate distally (e.g., be translated by a clinician) toward snare loop 126 to cause snare loop 126 (e.g., loop aperture 146) to constrict around IMD retrieval structure 111. For example, translation of snare sheath 130 toward snare loop 126 may cause some portion of snare loop 126 to enter sheath lumen 132, at least partially collapsing loop aperture 146 and constricting snare loop 126 around retrieval structure 111. In examples, snare 122 is configured such a proximal force exerted (e.g., by a clinician) on snare body proximal portion 123 (FIG. 1) and/or a distal force exerted (e.g., by a clinician) on snare sheath proximal portion 133 (FIG. 1) causes snare loop 126 to constrict around retrieval structure 111.

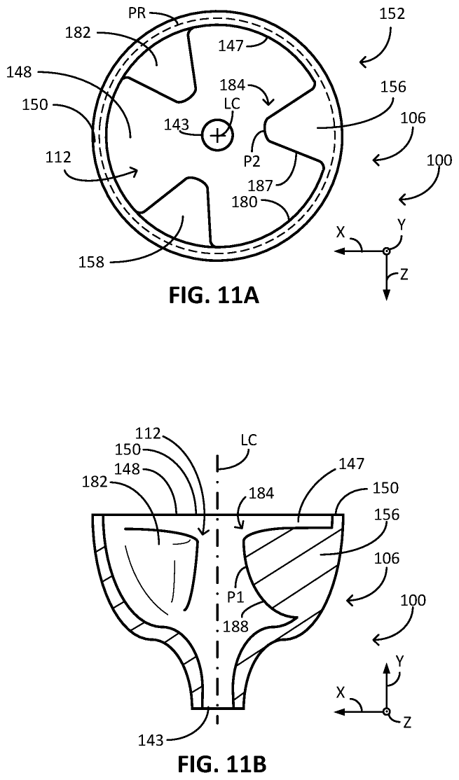

Snare body 124 and/or snare loop 126 may translate (e.g., within driver lumen 128 and/or sheath lumen 132) in the distal direction D and/or the proximal direction P relative to driver 102, delivery catheter 134, and/or IMD 110. Snare body 124 may be configured such that a force exerted on snare body 124 (e.g., exerted on snare body proximal portion 123, by a clinician) causes a translation of snare body 124 within driver lumen 128 and/or sheath lumen 132, and the translation of snare body 124 causes a translation of snare loop 126. Snare body 124 may be configured to alter a position of snare 122 relative to driver 102, delivery catheter 134, and/or IMD 110. For example, snare 122 may be translated (e.g., by a clinician exerting a force on snare body 124) in the distal direction D substantially toward IMD 110 to place snare loop 126 in proximity to IMD retrieval structure 111. Some portion of snare 122 (e.g., snare body 124) may be translated (e.g., by a clinician exerting a force on snare body 124) in the proximal direction P substantially away from IMD 110 to cause snare loop 126 to constrict around IMD retrieval structure 111. Snare 122 may be configured to translate within driver lumen 128 and/or sheath lumen 132 such that snare loop 126 positions either distal to or proximal to an opening 141 defined by delivery receptacle 140 (“delivery receptacle opening 141”). For example, snare loop 126 may position distal to delivery receptacle opening 141 (as depicted in FIGS. 2, 3) and/or may position proximal to delivery receptacle opening 141 (as depicted in FIG. 11).

Torque coupler 106 includes a coupler surface 147 at least partially defining coupler volume 112. Coupler surface 147 is configured such that torque coupler 106 may receive at least some portion of IMD retrieval structure 111 within coupler volume 112. In examples, torque coupler 106 defines an opening 148 (“coupler opening 148”) opening into coupler volume 112. Coupler opening 148 is sized such that at least a portion of IMD retrieval structure 111 may pass through coupler opening 148 when coupler volume 112 receives IMD retrieval structure 111. In examples, a distal end 150 of torque coupler 106 (“coupler distal end 150”) defines coupler opening 148. Torque coupler 106 (e.g., coupler surface 147) is configured such coupler volume 112 may receive at least a portion of IMD retrieval structure 111 when torque coupler 106 approaches IMD retrieval structure 111 along a path substantially offset (e.g., oblique to) IMD axis LD (e.g., along a path causing coupler axis LC and IMD axis LD to define an oblique angle such as angle A1). As will be discussed, driver 102 may be configured such that snare 122 substantially guides driver 102 (e.g., torque coupler 106) toward IMD retrieval structure when snare 122 engages IMD retrieval structure 111 and driver 102 translates over snare 122 (e.g., as snare 122 extends within driver lumen 128).

Coupler surface 147 defines a plurality of protrusions 154 (“protrusions 154”) including protrusion 156 and/or protrusion 158. Protrusions 156, 158 may extend within coupler volume 112 in a direction substantially toward coupler axis LC. Protrusions 156, 158 are configured to engage IMD retrieval structure 111 to transfer torque to IMD 110 when coupler volume 112 receives IMD retrieval structure 111 and a torque is exerted (e.g., by a clinician) on driver 102 (e.g., on driver body proximal portion 107 (FIG. 1)). In examples, IMD retrieval structure 111 defines a plurality of petals 160 including IMD petal 162 and/or IMD petal 164. Protrusions 156, 158 may be configured engage IMD petals 162, 164 to cause torque coupler 106 to transfer torque to IMD retrieval structure 111. In examples, protrusion 156, 158 may be configured to substantially slip over IMD petal 162, 164 as torque coupler 106 rotates relative to IMD retrieval structure 111, until at least one of protrusion 156 or protrusion 158 engages at least of IMD petal 162 or IMD petal 164 sufficiently to transfer torque to IMD retrieval structure 111. In examples, medical system 100 is configured to increase a contact force between coupler surface 147 and IMD retrieval structure 111 to cause protrusion 156, 158 to engage IMD petal 162, 164 sufficiently to transfer torque. For example, a clinician may exert a distal force on driver body 104 (e.g., driver body proximal portion 107 (FIG. 1)) and/or a proximal force on snare 122 (e.g., snare body proximal portion 123) to increase the contact force. The clinician may adjust the distal force and/or proximal force to adjust the contact force.

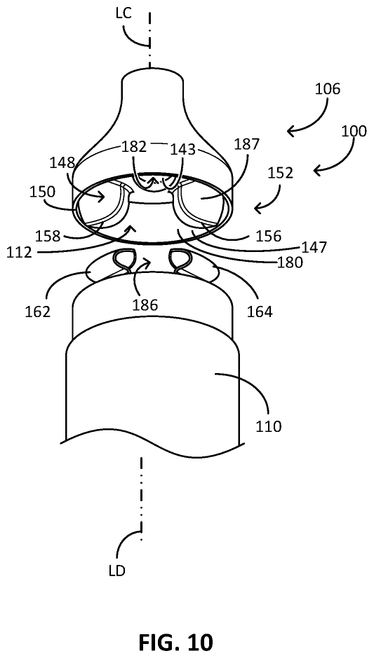

Driver body 104 may translate (e.g., within delivery catheter lumen 142) in the distal direction D and/or the proximal direction P relative to snare 122, delivery catheter 134, and/or IMD 110. Driver body 104 may be configured such that a force exerted on driver body 104 (e.g., exerted on driver body proximal portion 107, by a clinician) causes a translation of driver body 104 within delivery catheter lumen 142, and the translation of driver body 104 causes a translation of torque coupler 106. Driver body 104 may be configured to alter a position of torque coupler 106 relative to snare 122, delivery catheter 134, and/or IMD 110. For example, torque coupler 106 may be moved (e.g., by a clinician exerting a force on driver body 104) in the distal direction D substantially toward IMD 110 to cause coupler volume 112 to receive IMD proximal portion 114 (and/or snare 122). Torque coupler 106 may be moved (e.g., by a clinician exerting a force on driver body 104) in the proximal direction P substantially away from IMD 110 to cause IMD 110 (and/or snare 122) to exit coupler volume 112. Driver body 104 may be configured to translate within delivery catheter lumen 142 such that torque coupler 106 and/or other portions of driver body 104 position distal to and/or proximal to either or both of an opening 141 defined by delivery receptacle 140 (“delivery receptacle opening 141”) and/or snare 122. For example, torque coupler 106 may position distal to delivery receptacle opening 141 (as depicted in FIGS. 2, 3) and/or may position proximal to delivery receptacle opening 141 (as depicted in FIG. 10).

Delivery receptacle 140 may define delivery receptacle opening 141 at a distal end 139 of delivery receptacle 140 (“delivery receptacle distal end 139”) into delivery receptacle volume 168. Delivery receptacle opening 141 is sized such that at least some portion of snare 122, torque coupler 106, at least some portion of driver body distal portion 105, and/or at least some portion of IMD 110 (e.g., IMD retrieval structure 111) may pass in the proximal direction P and/or in the distal direction D through delivery receptacle opening 141. Delivery catheter lumen 142 is configured such that at least driver body 104 may translate within delivery catheter lumen 142 in the distal direction D and/or the proximal direction P.

Delivery receptacle 140 defines a delivery receptacle volume 168 configured to receive torque coupler 106, at least a portion of snare 122, and at least a portion of IMD 110. Delivery receptacle opening 141 and delivery catheter lumen 142 opens to delivery receptacle volume 168. Delivery receptacle 140 is configured such that at least torque coupler 106, at least a portion of snare 122, and at least a portion of IMD 110 may position within delivery receptacle volume 168 when snare 122 and/or torque coupler 106 is engaged with IMD 110. In examples, delivery receptacle 140 may be configured such that substantially an entirety of IMD 110 may position within delivery receptacle volume 168 when snare 122 and/or torque coupler 106 is engaged with IMD 110. For example, delivery catheter 134 may move in the distal direction D relative to snare 122, torque coupler 106, and/or IMD 110 to receive snare 122, torque coupler 106, and/or IMD 110. Delivery catheter 134 may move in the proximal direction P relative to snare 122, torque coupler 106, and/or IMD 110 to cause snare 122, torque coupler 106, and/or IMD 110 to position distal to delivery receptacle opening 141.

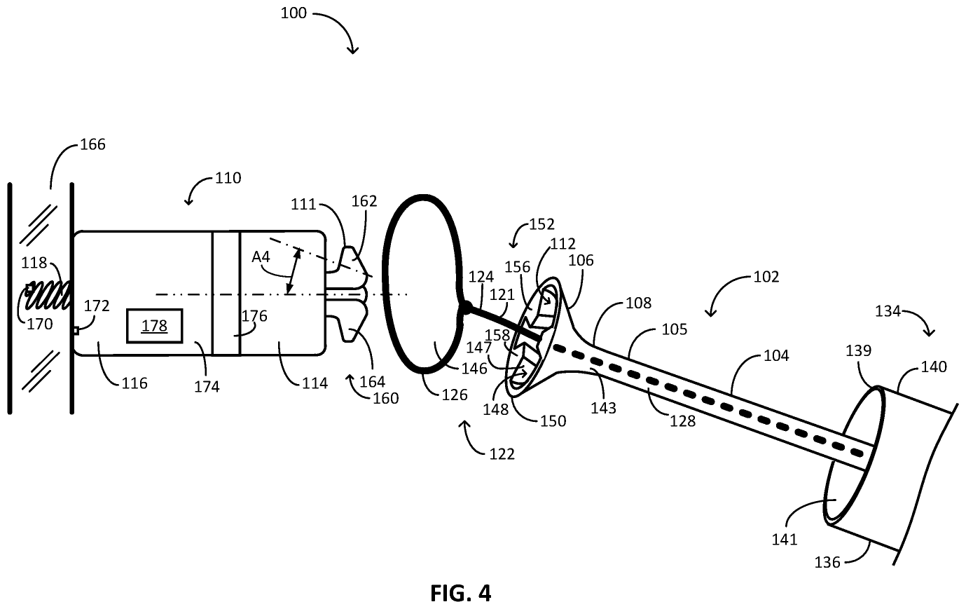

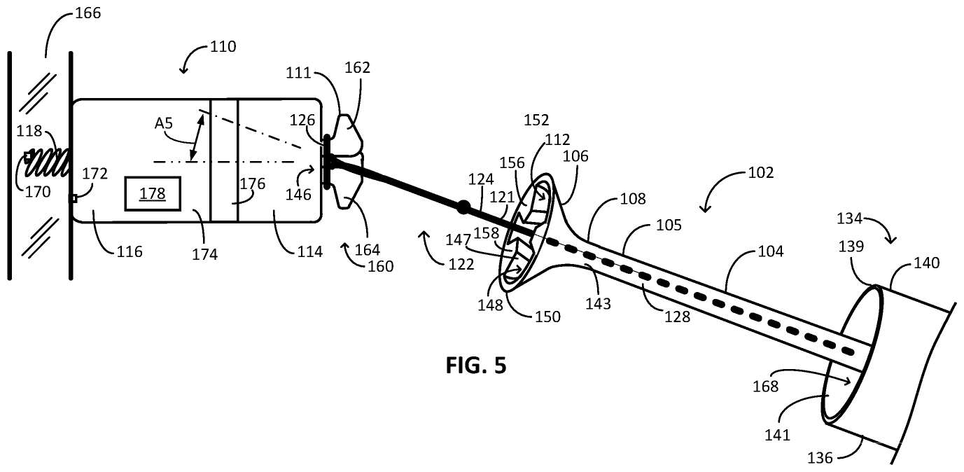

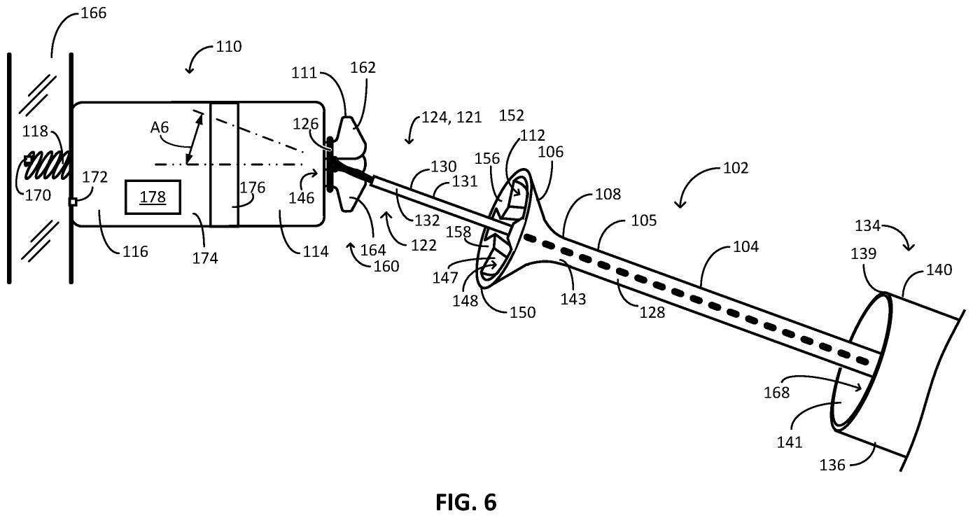

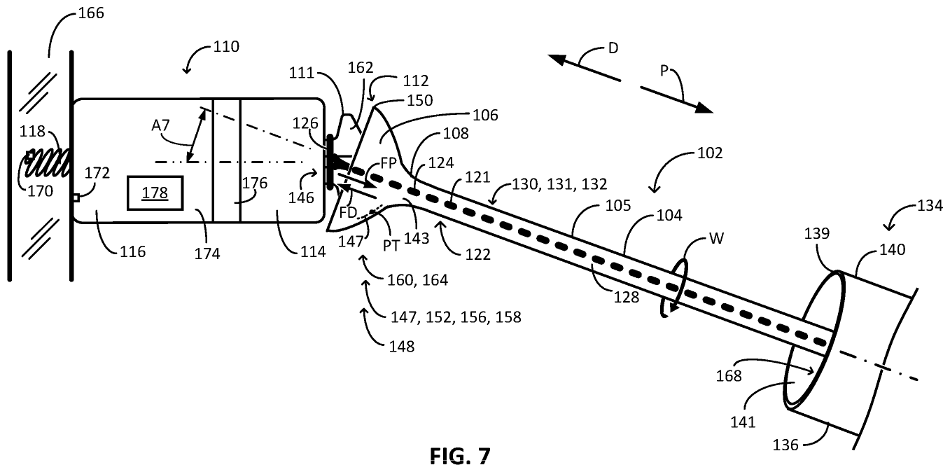

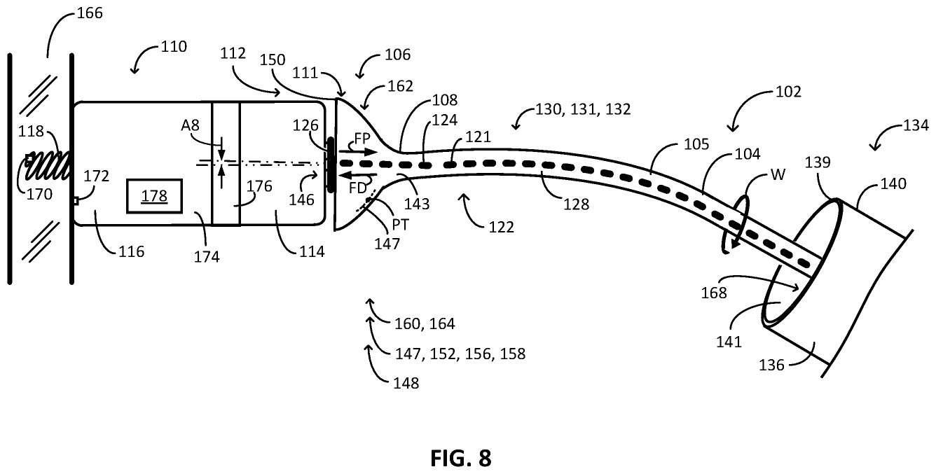

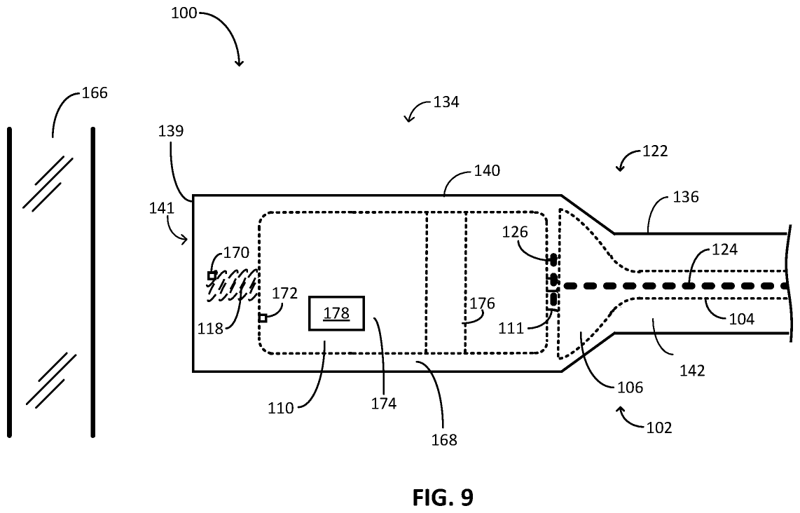

FIGS. 4-9 illustrate medical system 100 retrieving an IMD 110 secured to a tissue wall 166 by attachment member 118. In the examples of FIGS. 4-9, attachment member 118 defines a helix. In other examples, attachment member 118 may define other structures, such as one or more elongated tines. Tissue wall 166 may be within target site 120 (FIG. 1). Further, in the examples of FIGS. 4-8, torque coupler 106 is oriented relative to IMD 110 such that coupler axis LC and IMD axis LD define an oblique angle (e.g., angle A1 (FIGS. 2, 3)), although this is not required. Further, in the examples of FIGS. 4-8, torque coupler 106 is oriented relative to IMD 110 such that coupler axis LC and IMD axis LD define an oblique angle A4 (FIG. 4), an oblique angle A5 (FIG. 5), an oblique angle A6 (FIG. 6), an oblique angle A7 (FIG. 7), and an oblique angle A8 (FIG. 8). In examples, oblique angle A4, A5, A6, A7, A8 defines an angular displacement between coupler axis LC and IMD axis LD. In some examples, oblique angle A4, A5, A6, A7, A8 defines an angular displacement between coupler axis LC and a projection of IMD axis LD on a geometric plane including coupler axis LC. Any one of oblique angles A4, A5, A6, A7, A8 may describe substantially the same angular displacement or a different angular displacement as any other of oblique angles A4, A5, A6, A7, A8. Additionally, although not depicted, coupler axis LC and IMD axis LD may define an oblique angle in FIG. 9.

FIG. 4 illustrates snare 122 extended distal to torque coupler 106 to engage retrieval structure 111 of IMD 110. Snare 122 (e.g., snare loop 126 and/or snare body 124) may be configured to engage IMD retrieval structure 111 when coupler axis LC and IMD axis LD define oblique angle A4. Snare 122 may be configured such that a force (e.g., a distal force) exerted on snare body 124 (e.g., exerted on snare body proximal portion 123, by a clinician) causes a translation of snare loop 126 toward IMD retrieval structure 111. Snare body 124 may be sufficiently rigid to cause snare loop 126 to extend distally beyond coupler opening 148, such that snare loop 126 is positioned in proximity to IMD retrieval structure 111. In examples, snare loop 126 and/or snare body 124 may be resiliently biased such that snare loop 126 substantially establishes a particular orientation relative to snare body 124 when snare loop 126 positioned distal to torque coupler 106 and/or driver lumen 128 (e.g., when snare loop is substantially unconstrained by torque coupler 106 and/or driver lumen 128). For example, snare loop 126 and/or snare body 124 may be resiliently biased such that snare loop 126 and snare body 124 define an angle (e.g., an angle of about 90 degrees, about 45 degrees, or some other angle) when snare loop 126 is unconstrained by torque coupler 106 and/or driver lumen 128. Snare body 124 may be translated (e.g., by a clinician) through driver lumen 128 to cause loop aperture 146 of snare loop 126 to receive (e.g., substantially surround) at least some portion of IMD 110, such as IMD retrieval structure 111.

FIG. 5 illustrates snare loop 126 engaging retrieval structure 111 such that loop aperture 146 substantially surrounds IMD retrieval structure 111. In examples, snare 122 is configured such that, when snare loop 126 engages retrieval structure 111, proximal translation of snare body 124 through driver lumen 128 causes aperture 146 to constrict around retrieval structure 111. In FIG. 5, with snare loop 126 engaging retrieval structure 111, coupler axis LC and IMD axis LD define oblique angle A5, which may describe an angular displacement substantially similar to or different from oblique angle A4. In some examples, as illustrated in FIG. 6, medical system 100 includes snare sheath 130. Snare sheath 130 may be translated relative to snare loop 126 to in the constriction of snare loop 126 around retrieval structure 111. In FIG. 6, with snare sheath translated relative to snare loop 126, coupler axis LC and IMD axis LD define oblique angle A6, which may describe an angular displacement substantially similar to or different from oblique angle A4, A5. In examples, snare 122 and/or snare sheath 130 are configured such that, when snare 122 is engaged with IMD retrieval structure 111, snare 122 and/or snare sheath 130 may substantially guide torque coupler 106 toward IMD retrieval structure to cause contact between torque coupler 106 and IMD retrieval structure (e.g., when driver 102 proximally translates as snare 122 and/or snare sheath 130 extend through driver lumen 128).

FIG. 7 illustrates driver 102 translated over snare loop 126 and/or snare sheath 130 to cause contact between torque coupler 106 and IMD retrieval structure 111. With torque coupler 106 contacting IMD retrieval structure 111, coupler axis LC and IMD axis LD define oblique angle A7, which may describe an angular displacement substantially similar to or different from oblique angle A4, A5, A6.

In FIG. 7, snare 122 exerts a proximally-directed force FP on retrieval structure 111 (e.g., caused by a clinician exerting force in the proximal direction on snare body proximal portion 123 (FIG. 1)). Torque coupler 106 exerts a distally-directed force FD on retrieval structure 111 (e.g., caused by a clinician exerting the distally-directed force FD on driver body proximal portion 107 (FIG. 1)). When torque coupler 106 is in contact with IMD retrieval structure 111, proximally-directed force FP and/or distally-directed force FD generate a contact force between torque coupler 106 (e.g., coupler surface 147) and IMD retrieval structure 111 (e.g., one or more of plurality of petals 160). Medical system 100 is configured such that increasing or decreasing proximally-directed force FP (e.g., by the clinician) may increase or decrease, respectively, the contact force between torque coupler 106 and IMD retrieval structure 111. Medical system 100 is configured such that increasing or decreasing distally-directed force FD (e.g., by the clinician) may increase or decrease, respectively, the contact force the contact force between torque coupler 106 and IMD retrieval structure 111. Hence, medical system 100 is configured such that a clinician may vary and/or alter the contact force between torque coupler 106 and IMD retrieval structure 111 by varying and/or altering the proximally-directed force FP and/or the distally-directed force FD.

Torque coupler 106 may be caused to rotate (e.g., by a clinician) relative to IMD retrieval structure 111 (e.g., when coupler axis LC and IMD axis LD define oblique angle A7). For example, FIG. 7 illustrates driver 102 experiencing a torque (e.g., imparted by a clinician via driver body proximal portion 107 (FIG. 1)) causing a rotation of driver body 104 and torque coupler 106 in a rotational direction W. The rotational direction W may be, for example, first rotational direction W1 or second rotational direction W2 (FIG. 1). Protrusions 154 (e.g., protrusion 156, 158) of coupler surface 147 may be configured to slidably translate over (e.g., slip over) IMD retrieval structure 111 (e.g., IMD petal 162, 164) when torque coupler 106 contacts IMD retrieval structure 111, torque coupler 106 rotates in the rotational direction W, and the contact force (caused by proximally-directed force FP and/or distally-directed force FD) provides a slipping force. In examples, protrusion 152 are configured to rotate around coupler axis LC to slidably translate over IMD retrieval structure 111.

Protrusions 154 may slip over IMD retrieval structure 111 until the contact force provides an engagement force causing at least one of protrusions 154 (e.g., at least one of protrusion 156 or protrusion 158) to transfer torque causing a rotation of IMD 110 (e.g., a rotation of IMD 110 about IMD axis LD).

The contact force may be increased (e.g., by the clinician) from a slipping force to cause at least one of protrusion 156 or protrusion 158 to sufficiently engage IMD retrieval structure 111 for the transfer of torque sufficient to cause the rotation of IMD 110. For example, as torque coupler 106 contacts and rotates relative to IMD retrieval structure 111, a clinician may increase the proximally-directed force FP and/or increase the distally-directed force FD to increase the contact force. The clinician may continue to controllably increase the contact force until the contact force provides an engagement force, such that at least one of protrusion 156 or protrusion 158 transfers torque sufficiently to cause rotation of IMD 110. The clinician may substantially maintain and/or adjust the contact force as protrusion 156, 158 transfers torque to the IMD retrieval structure 111, such that medical system 100 may transfer torque when coupler axis LC of torque coupler 106 and the IMD axis of the IMD define oblique angle A7. Hence, torque coupler 106 may be configured to engage and/or transfer torque to an IMD when torque coupler 106 approaches the IMD along a path substantially offset (e.g., oblique to) an IMD axis of the IMD.

Medical system 100 may be configured to control (e.g., increase, decrease, and/or substantially maintain) the contact force to improve and/or control the torque transfer from torque coupler 106 to IMD retrieval structure 111. For example, as illustrated at FIG. 8, medical system 100 may be configured such that adjustments to distally-directed force FD and/or proximally-directed force FP, and/or continued rotation of torque coupler 106 relative to IMD retrieval structure 111, causes torque coupler 106 and/or IMD 110 to reduce the oblique angle defined between coupler axis LC and IMD axis LD (e.g., reduce the oblique angle from oblique angle A7 (FIG. 7) to oblique angle A8).

In examples, torque coupler 106 is configured to reduce the oblique angle as distally-directed force FD and proximally-directed force FP act on IMD retrieval structure 111. For example, when torque coupler 106 contacts IMD retrieval structure 111, the contact may establish a pivot point PT (FIG. 7, FIG. 8) on coupler surface 147 about which torque coupler 106 may pivot and/or rock relative to IMD retrieval structure 111. Torque coupler 106 may be configured such that contact force (e.g., as controlled by a clinician) between torque coupler 106 and IMD retrieval structure 111 causes torque coupler 106 to substantially pivot about pivot point PT in a direction toward IMD retrieval structure 111, reducing the oblique angle between coupler axis LC and IMD axis LD. In some examples, rather than or in addition to pivoting about pivot point PT, torque coupler 106 may be configured such that the contact force causes coupler surface 147 to slidably translate over IMD retrieval structure 111 in a direction causing a reduction of the oblique angle. For example, at least a portion of coupler surface 147 may configured to conform with a surface defined by IMD petal 162, 164. Torque coupler 106 may be configured such that the contact force causes the portion of coupler surface 147 to slidably translate over the surface defined by IMD petal 162, 164 to cause a reduction of the oblique angle. In examples, as illustrated in FIG. 8, driver body 104 may be configured to flex and/or bend (e.g., define a curved or curvilinear shape) when torque coupler 106 pivots and/or slidably translates to reduce the oblique angle between coupler axis LC and IMD axis LD. Driver body 104 and torque coupler 106 may be configured to rotate in the rotational direction W when driver body 104 flexes and/or bends.

FIG. 9 is a schematic illustration of snare 122, torque coupler 106, and IMD 110 positioned within delivery receptacle volume 168 defined by delivery receptacle 140. Delivery catheter 134 may move in the distal direction D relative to snare 122, torque coupler 106, and/or IMD 110 to cause snare 122, torque coupler 106, and/or IMD 110 to position in delivery receptacle volume 168. For example, delivery receptacle volume 168 may receive snare 122, torque coupler 106, and/or IMD 110 subsequent to attachment member 118 disengaging from tissue wall 166, such that delivery catheter 134 may withdraw snare 122, torque coupler 106, and/or IMD 110 from a patient (e.g., via vasculature of the patient). Likewise, delivery catheter 134 may move in the proximal direction P relative to snare 122, torque coupler 106, and/or IMD 110 to cause snare 122, torque coupler 106, and/or IMD 110 to exit and/or position distal to delivery receptacle volume 168. For example, delivery catheter 134 may cause snare 122, torque coupler 106, and/or IMD 110 to exit and/or position distal to delivery receptacle volume 168 subsequent to delivery of snare 122, torque coupler 106, and/or IMD 110 (e.g., via vasculature of a patient) to an anatomical volume of the patient.

IMD 110, which in some examples can comprise a pacemaker such as a leadless and/or wholly intracardiac pacemaker, may include one or more electrodes such as electrode 170 supported by attachment member 118, electrode 172 supported by a housing 174 of IMD 110 (“IMD housing 174”), and/or electrode 176 (e.g., a return electrode) supported by IMD housing 174. One or more of electrodes 170, 172, 176 may be electrically connected to operating circuitry 178. Operating circuitry 178 may be configured to deliver therapy to a patient and/or sense physiological signals of the patient using electrodes 170, 172, 176. In examples, at least a portion of operating circuitry 178 is supported by IMD housing 174. In some examples, at least a portion of operating circuitry 178 is supported by another device displaced from IMD 110, such as another device within the patient and/or another device extracorporeal to the patient.

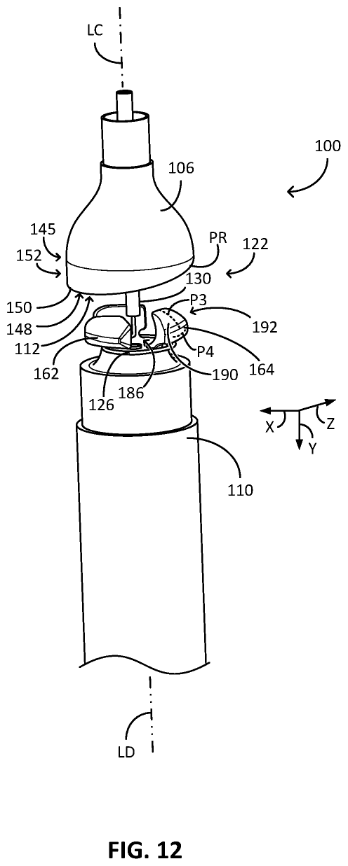

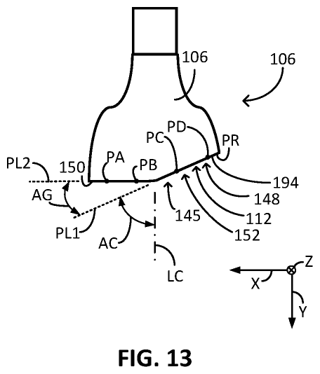

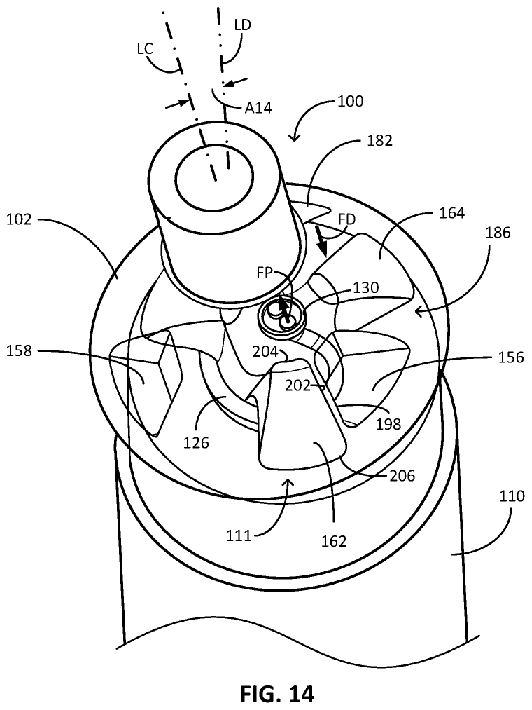

FIG. 10 is an perspective view illustrating torque coupler 106 and a portion of IMD 110 including retrieval structure 111. FIG. 11A is a schematic top view of torque coupler 106, with coupler axis LC and a y axis of the x-y-z axes shown proceeding out of the page. FIG. 11B is an schematic cross-section view of torque coupler 106 with a cutting plane taken through coupler axis LC, and a z axis of the x-y-z axes shown proceeding out of the page. FIG. 12 is an perspective view illustrating an example torque coupler 106 and IMD 110, with snare 122 engaging retrieval structure 111. FIG. 13 is a schematic plan view of an example torque coupler 106. The y axis is parallel to coupler axis LC in FIG. 11A, FIG. 11B, and FIG. 13, and parallel to coupler axis LC and device axis LD in FIG. 12. The y axis proceeds out of the page in FIG. 11A, the z axis proceeds out of the page in FIG. 11B, and the z axis proceeds into the page in FIG. 12 and FIG. 13. Torque coupler 106 includes coupler surface 147 defining protrusions 154, including protrusion 156 (e.g., a first protrusion), protrusion 158 (e.g., a second protrusion) and a base portion 180 separating protrusion 156 and protrusion 158. Protrusions 154 may include one or more additional protrusions, such as protrusion 182.

Coupler surface 147 may define coupler volume 112. In examples, coupler surface 147 defines a boundary of coupler volume 112 extending substantially from driver lumen opening 143 to coupler opening 148. Coupler surface 147 may be configured (e.g., shaped with respect to coupler axis LC) to allow and/or promote contact between coupler surface 147 and IMD retrieval structure 111 when coupler axis LC and IMD axis LD define an oblique angle such as oblique angle A1, A4, A5, A6, A7, A8. Coupler surface 147 may be configured such that a contact force (e.g., caused by proximally-directed force FP and/or distally directed force FD (FIGS. 7, 8) causes torque coupler 106 to pivot and/or slidably translate relative to IMD retrieval structure 111 to reduce the oblique angle. In examples, at least a portion of coupler surface 147 (e.g., base portion 180) defines a concavity (e.g., in the x-y plane, x-z plane, or y-z plane) curving inward toward coupler axis LC. In examples, at least a portion of coupler surface 147 (e.g., base portion 180) increasingly expands radially outward from coupler axis LC as the portion of coupler surface 147 extends in a direction from driver lumen opening 143 toward coupler opening 148.

Protrusion 156, 158, 182 may be configured to slip over IMD retrieval structure 111 until at least one of protrusion 156, 158, 162 engages IMD retrieval structure 111 sufficiently to transfer a torque causing a rotation of IMD 110 (e.g., until a contact force from torque coupler 106 to IMD retrieval structure 111 provides an engagement force). In some examples, protrusion 156, 158, 182 defines one or more rounded corners to assist protrusion 156, 158, 182 in slidably translating over IMD retrieval structure 111. A rounded corner may define a curved, curvilinear, and/or polygonal curved profile in a geometric plane substantially parallel to, perpendicular to, or including coupler axis LC. For example, protrusion 156 includes rounded corner 184. Rounded corner 184 defines curved, curvilinear, and/or polygonal curved profile P1 (FIG. 11B) in a geometric plane defined by the x axis and the y axis and substantially parallel to or including coupler axis LC. Rounded corner 184 defines curved, curvilinear, and/or polygonal curved profile P2 (FIG. 11A) in a geometric plane defined by the x axis and the z axis and perpendicular to coupler axis LC. At least one protrusion (e.g., protrusion 156) may define a convexity (e.g., in the x-y plane, x-z plane, or y-z plane) curving outward away from coupler axis LC (e.g., curved, curvilinear, and/or polygonal curved profile P1, P2).

In examples, protrusion 156, 158, 182 may be configured (e.g., shaped with respect to coupler axis LC) to allow and/or promote an engagement with IMD retrieval structure 111 when coupler axis LC and IMD axis LD define oblique angle A1, A4, A5, A6, A7, A8, and/or another oblique angle. For example, protrusion 156, 158, 182 may be configured to allow and/or promote the insertion of protrusion 156, 158, 182 within a recess defined by IMD retrieval structure 111 between two or more IMD petals, such as IMD recess 186 defined between IMD petal 162 and IMD petal 164. In examples, at least a portion of protrusion 156, 158, 182 (e.g., portion 188 of protrusion 156) increasingly contracts radially inward from coupler axis LC as the portion of protrusion 156, 158, 182 extends in a direction from coupler opening 148 to driver lumen opening 143.

Coupler surface 147 may include a plurality of protrusion surfaces defining protrusions 154, such as protrusion surface 187 of protrusion 156. In examples, protrusion surface 187 and base portion 180 form a contiguous portion of coupler surface 147. For example, protrusion surface 187 and base portion 180 may be joined to define a curved portion of coupler surface 147, a cornered portion of coupler surface 147, a substantially planar portion of coupler surface 147, or a portion of coupler surface 147 exhibiting another surface profile. In examples, at least a portion of protrusion surface 187 and/or at least a portion of base portion 180 define a curved, curvilinear, and/or polygonal curved profile in a geometric plane substantially parallel to, perpendicular to, or including coupler axis LC.

Coupler distal end 150 may be configured to assist and/or promote the insertion of protrusion 156, 158, 182 within IMD recess 186 when protrusion 156, 158, 182 slidably translates around IMD retrieval structure 111 (e.g., when torque coupler 106 contacts and rotates relative to IMD retrieval structure 111). In examples, torque coupler 106 defines coupler distal end 150 such that an end coupler perimeter PR (FIGS. 11A, 12, 13) surrounding coupler opening 148 is substantially non-planar. A non-planar coupler opening 148 may allow protrusion 156, 158, 182 to insert within IMD recess 186 more readily during torque coupler 106 rotation when, for example, coupler axis LC and IMD axis LD define oblique angle A1, A4, A5, A6, A7, A8, and/or another oblique angle.

End coupler perimeter PR may include one or more curved segments, curvilinear segments, polygonal curved segments, and/or other segments. In examples, end coupler perimeter PR is non-planar. For example, end coupler perimeter PR may include a plurality of non-coplanar points such as PA, PB, PC, and PD. In some examples, torque distal end 150 defines end coupler perimeter PR such that at least a portion 194 of end coupler perimeter PR (““perimeter portion 194”) defines an acute angle AC with respect to coupler axis LC. For example, perimeter portion 194 may define a plane PL1 angularly displaced from coupler axis LC by the acute angle AC. Acute angle AC may define any acute angular displacement from coupler axis LC. In examples, acute angle AC defines an angular displacement of less than about 80 degrees, less than about 70 degrees, less than about 60 degrees, or less than about 45 degrees. In some examples end coupler perimeter PR includes a second portion 196 (“second perimeter portion 196”) defining a plane PL2. Torque coupler 106 may define perimeter PR such that plane PL1 and plane PL2 are angularly displaced by an angle AG, such that perimeter portion 194 and second perimeter portion 196 are substantially non-coplanar and/or non-parallel. Angle AG may define any angular displacement between perimeter portion 194 and second perimeter portion 196. In examples, angle AG defines an angular displacement of greater than about 10 degrees, greater than about 20 degrees, greater than about 30 degrees, or greater than about 45 degrees. In some examples, torque coupler 106 defines coupler distal end 150 such that substantially an entirety of end coupler perimeter PR defines plane PL1.

In examples, IMD petal 162, 164 extends in a direction substantially radial to IMD axis LD. IMD petal 162, 164 may be configured to allow and/or promote the slidable translation of protrusion 156, 158, 182 over IMD retrieval structure 111 until at least one of protrusion 156, 158, 162 engages IMD retrieval structure 111 to transfer a torque causing a rotation of IMD 110 (e.g., until a contact force from torque coupler 106 to IMD retrieval structure 111 provides an engagement force). In examples, IMD petal 162, 164 defines one or more lobes such as lobe 190 (FIG. 12) configured to allow and/or assist the slidable translation of protrusion 156, 158, 182 over IMD retrieval structure 111. Lobe 190 may include one or more rounded corners defining a curved, curvilinear, and/or polygonal curved profile in a geometric plane substantially parallel to, perpendicular to, or including device axis LD. For example, lobe 190 may include rounded corner 192. Rounded corner 192 defines curved, curvilinear, and/or polygonal curved profile P3 in a geometric plane defined by the x axis and the y axis and substantially parallel to or including device axis LD. Rounded corner 192 defines curved, curvilinear, and/or polygonal curved profile P4 in a geometric plane defined by the x axis and the z axis and perpendicular to coupler axis LC. In examples, at least one IMD petal (e.g., IMD petal 164) may define a concavity (e.g., in the x-y plane, x-z plane, or y-z plane) curving inward toward device axis LD.

As discussed, protrusions 154 are configured such that at least one of protrusion 156, 158, 182 transfers a torque from torque coupler 106 to IMD retrieval structure when a proximally-directed force FP (FIG. 7, 8) exerted by snare 122 and a distally-directed force FD (FIG. 7, 8) exerted by torque coupler 106 generate an engagement force as protrusion 156, 158, 182 contacts IMD retrieval structure 111. The engagement force is a contact force having a having a force magnitude sufficient to cause protrusion 156, 158, 182 to transfer a torque to IMD retrieval structure 111 sufficient to, for example, cause a rotation of IMD 110 about IMD axis LD. In examples, the engagement force cause protrusion 156, 158, 182 to transfer a torque to IMD retrieval structure 111 when torque coupler 106 rotates relative to IMD retrieval structure 111 and protrusion 156, 158, 182 inserts into IMD recess 186.

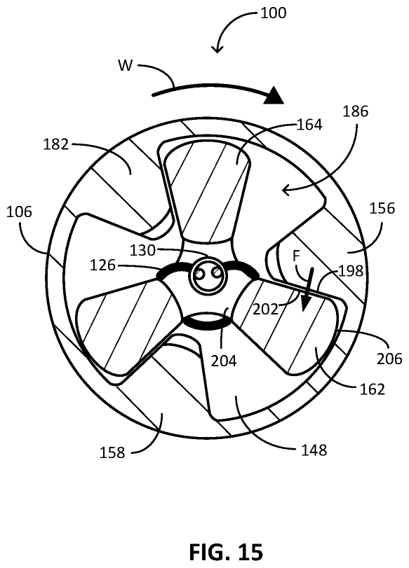

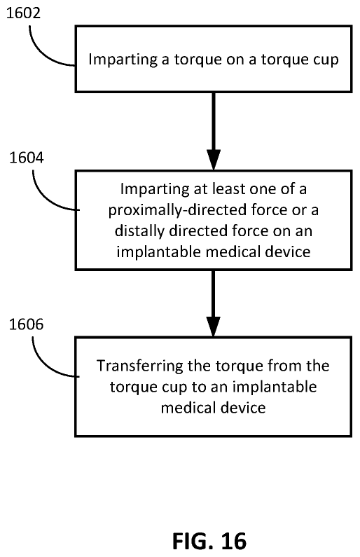

For example, FIG. 14 is a perspective view of an example torque coupler 106 with protrusion 156 inserted within IMD recess 186 of IMD retrieval structure 111. Coupler axis LC and IMD axis LD define an oblique angle A14, which may any of oblique angle A1, A4, A5, A6, A7 and/or another oblique angle. Torque coupler 106 is illustrated as transparent in FIG. 14 for clarity. FIG. 15 is a schematic cross-sectional view of torque coupler 106 and IMD retrieval structure 111, with a cutting plane taken perpendicular to coupler axis LC. FIGS. 14, 15 may be representative of the respective orientations of torque coupler 106 and IMD retrieval structure when coupler axis LC and IMD axis LD define an oblique angle such as oblique angle A8 (FIG. 7).

Snare 122 (e.g., snare loop 126) is engaged with IMD retrieval structure 111, such that snare 122 may exert proximally-directed force FP on IMD 110 (e.g., IMD retrieval structure 111). In examples, snare sheath 130 causes a constriction of snare loop 126 around IMD retrieval structure 111. Torque coupler 106 (e.g., coupler surface 147) contacts IMD retrieval structure 111, such that torque coupler 106 may exert distally-directed force FP on IMD retrieval structure 111. Snare 122 and torque coupler 106 are oriented with respect to IMD 110 such that the proximally-directed force FP and/or the distally-directed force FD generate a contact force exerted from torque coupler 106 to IMD retrieval structure 111.

As discussed, protrusion 156 is configured to slidably translate over (e.g., slip over) IMD retrieval structure 111 when torque coupler 106 contacts IMD retrieval structure 111, torque coupler 106 rotates in the rotational direction W, and the contact force provides a slipping force. Protrusion 156 may slip over IMD retrieval structure 111 until the contact force provides an engagement force sufficient to cause protrusion 156 to transfer torque causing a rotation of IMD 110. Proximally-directed force FP and/or distally-directed force FD may be altered (e.g., increased) to substantially cease the slippage of protrusion 156 over IMD retrieval structure 111 (e.g., over IMD petal 162, 164) and cause protrusion 156 to transfer the torque to IMD retrieval structure 111. For example, a clinician may alter a force in a proximal direction on snare body proximal portion 123 (FIG. 1) to alter proximally-directed force FP. The clinician may alter a force in a distal direction on driver body proximal portion 107 (FIG. 1) to alter proximally-directed force FP. The clinician may alter the proximally-directed force FP and/or distally-directed force FD such that the contact force provides an engagement force, and such that protrusion 156 transfers a torque causing rotation of IMD 110. For example, protrusion 156 may be configured to transmit a force F to IMD retrieval structure 111 (e.g., IMD petal 162) when torque coupler 106 experiences a torque in the rotational direction W and proximally-directed force FP and/or distally-directed force FD cause the contact force to provide an engagement force.

In examples, protrusion 156 includes a bearing surface 198 (“protrusion bearing surface 198”) configured to contact IMD petal 162 when protrusion 156 inserts into IMD recess 186. Protrusion 156 may be configured such that protrusion bearing surface 198 extends substantially radially inward toward coupler axis LC. Protrusion 156 may be configured to substantially distribute the force F over protrusion bearing surface 198 when protrusion 156 transmits force F to IMD retrieval structure 111 (e.g., IMD petal 162). In some examples, protrusion bearing surface 198 is configured to contact a bearing surface 202 of IMD petal 162 (“IMD bearing surface 202”) when protrusion 156 inserts into IMD recess 186.

IMD bearing surface 202 may be configured to receive the force F from protrusion 156 as a distributed force over IMD bearing surface 202. IMD petal 162 may be configured such that IMD bearing surface 202 extends substantially radially outward from device axis LD. In examples, IMD includes a petal root 204 and a petal distal end 206 extending substantially radially outward device axis LD. IMD petal 162 may be configured such that IMD bearing surface 202 extends between and/or substantially separates petal root 204 and petal distal end 206. Hence, IMD petal 162 may be configured to receive force F from protrusion 156 over an area extending radially outward toward petal distal end 206, rather than, for example, receiving force F as a substantially concentrated force generally in the area of petal distal end 206.

In examples, at least some portion of protrusion bearing surface 198 is configured to be substantially parallel and/or conforming to at least some portion of IMD bearing surface 198 when protrusion 156 inserts into IMD recess 186. Protrusion bearing surface 198 may be configured to contact IMD bearing surface 198 when coupler axis LC and device axis LD define an oblique angle such as one or more of oblique angle A1, A4, A5, A6, A7, A14, such that torque coupler 106 may transfer torque to IMD 110 when torque coupler 106 is axially offset (e.g., oblique to) IMD 110. In examples, protrusion bearing surface 198 is slidably translatable over IMD bearing surface 198 when protrusion 156 inserts into IMD recess 186, such that protrusion bearing surface 198 may transfer the force F to IMD bearing surface 202 over a range of angular displacements defined by oblique angle A14.

As discussed, driver body 104 is configured to transmit a torque to torque coupler 106 (e.g., a torque around coupler axis LC) to cause a rotation of torque coupler 106 (e.g., about coupler axis LC). Driver body 104 may be mechanically connected to torque coupler 106 in any manner which establishes a rotational coupling between driver body 104 and torque coupler 106, such as by welding, soldering, adhesives, pins, or some other suitable fastening method. In some examples, driver body 104 includes torque coil. The torque coil may have the form of a helix substantially surrounding a helix interior. Medical system 100 may be configured such that coupler axis LC passes through at least some portion of the helix interior. In some examples, the helix interior defines at least part of driver lumen 128. In examples, driver body 104 and/or snare sheath 130 may include a polymer material, such as a medical-grade polymer.

Operating circuitry 178 may include fixed function circuitry and/or programmable operating circuitry. In examples, operating circuitry 178 may include circuitry configured to perform one or more functions of operating circuitry 178, such as therapy delivery circuitry, sensing circuitry, processing circuitry, switching circuitry, communication circuitry, and/or other circuitries. Operating circuitry 178, as well as other processors, processing circuitry, controllers, control circuitry, and the like, described herein, may include any combination of integrated circuitry, discrete logic circuitry, analog circuitry, such as one or more microprocessors, digital signal processors (DSPs), application specific integrated circuits (ASICs), or field-programmable gate arrays (FPGAs). In some examples, operating circuitry 178 includes multiple components, such as any combination of one or more microprocessors, one or more DSPs, one or more ASICs, or one or more FPGAs, as well as other discrete or integrated logic circuitry, and/or analog circuitry.

Functions attributed to operating circuitry 178 may be embodied as software, firmware, hardware or any combination thereof. Operating circuitry 178 may include, for instance, a variety of capacitors, transformers, switches, and the like configured to perform the functions of operating circuitry 178. In examples, operating circuitry 178 may be configured to communicate with another device, such as a patient input/output device, a clinician input/output device, and/or others. Operating circuitry 178 may include any suitable hardware, firmware, software or any combination thereof for communicating with another device. In addition, operating circuitry 178 may communicate with a networked computing device and a computer network. In examples, operating circuitry 178 and/or other circuitry of medical system 100 is configured to deliver stimulation signals to and/or receive sensing signals from electrodes 170, 172, 176 and/or other electrodes and/or sensors within medical system 100 or external to medical system 100. Operating circuitry 178 may be configured to provide electrical signals, e.g., pacing therapy, to electrodes 170, 172, 176. Operating circuitry 178 may be configured to receive electrical signals, e.g., sensed cardiac electrical signals, from electrodes 170, 172, 176.

Medical system 100 (e.g., operating circuitry 178) can also include memory configured to store program instructions, such as software, which may include one or more program modules, which are executable by operating circuitry 178. The program instructions may be embodied in software and/or firmware. The memory can include any volatile, non-volatile, magnetic, optical, or electrical media, such as a random access memory (RAM), read-only memory (ROM), non-volatile RAM (NVRAM), electrically-erasable programmable ROM (EEPROM), ferroelectric RAM (FRAM), flash memory, or any other digital media. In some examples, the memory includes computer-readable instructions that, when executed by operating circuitry 178 cause operating circuitry 178 to perform various functions described herein and/or other functions of operating circuitry 178.

IMD housing 174 may enclose operating circuitry 178 and/or other circuitry within medical system 10. IMD housing 174 may be configured to fluidly isolate operating circuitry 178 and/or other circuitry from an environment in contact with an exterior surface of IMD housing 174. In examples, IMD housing 174 is configured to hermetically seal an enclosure defined by IMD 110 and holding operating circuitry 178 and/or other circuitry. IMD housing 174 may be configured to define shapes that are easily accepted by the patient's body while minimizing patient discomfort. For example, IMD housing 174 may define a substantially cylindrical shape with cylindrical sidewalls. In other examples, IMD housing 174 may define substantially rectangular or other non-cylindrical shapes. IMD housing 174 may define shapes in which corners and edges are designed with relatively large radii, in order to present a housing having smoothly contoured exterior surfaces. In examples, attachment member 118 is coupled to IMD housing 174.

As used here, when a first portion of a system (e.g., medical system 100) supports a second portion of the system, this means that when the second portion causes a first force to be exerted on the first portion, the first portion causes a second force to be exerted on the second portion in response to the first force. The first force and/or second force may be a contact force and/or an action-at-a-distance force. For example, first force and/or second force may be mechanical force, a magnetic force, a gravitational force, or some other type of force. The first portion of the system may be a portion of the system or a portion of a component of the system. The second portion of the system may be another portion of the system or another portion of the same component or a different component. In some examples, when the first portion of the system supports the second portion of the system, this may mean the second portion is mechanically supported by and/or mechanically connected to the first portion.

A technique for imparting a torque using a medical system 100 is illustrated in FIG. 14. Although the technique is described mainly with reference to medical system 100 of FIGS. 1-15, the technique may be applied to other medical systems in other examples.

The technique includes imparting a torque, using a driver body 104 of a driver 102, on a torque coupler 106 supported at a driver body distal end 108 (1602). Driver body 104 may impart the torque on torque coupler 106 to cause torque coupler 106 to rotate within an anatomical volume (e.g., a heart chamber) defined by a patient. Torque coupler 106 may receive an IMD retrieval structure 111 within a coupler volume 112 defined by a coupler surface 147 of torque coupler 106. The torque may cause a rotation of a plurality of protrusions 154 defined by coupler surface 147 and extending toward a coupler axis LC extending through coupler volume 112. In examples, coupler volume 112 receives retrieval structure 111 when coupler axis LC defines an oblique angle with an IMD axis LD of an IMD 110.

The technique may include imparting at least one of a proximally directed force FP on the IMD or a distally-directed force FD on IMD retrieval structure generate an engagement force from torque coupler 106 to IMD retrieval structure 111 (1604). Snare 122 may engage IMD 110 to exert proximally-directed force FP. In examples, a snare loop 126 supported by a snare body 124 engages IMD 110 (e.g., IMD retrieval structure) to exert proximally-directed force FP. Snare loop 126 may constrict around IMD 110 to engage IMD 110. In examples, a snare sheath 130 is translated (e.g., translated distally) relative to snare body 124 to constrict snare loop 126 around IMD 110. Snare loop 126 may exert proximally-directed force FP on IMD 110 when a proximal force is exerted (e.g., by a clinician) on a snare body proximal portion 123. In examples, snare body 124 extends through driver lumen 128 and a driver lumen opening 143 when snare 122 engages IMD 110. Snare 122 may cause coupler volume 112 to receive IMD 110 (e.g., IMD retrieval structure 111) when proximally-directed force FP is exerted. In examples, IMD 110 moves proximally (e.g., in a proximal direction P) relative to torque coupler 106 to cause coupler volume 112 to receive IMD 110. In examples, torque coupler 106 moves distally (e.g., in a distal direction D) relative to IMD 110 to cause coupler volume 112 to receive IMD 110.

Torque coupler 106 may engage (e.g., contact) IMD retrieval structure 111 to exert distally-directed force FD. Torque coupler 106 may engage IMD retrieval structure 111 when coupler volume 112 receives IMD retrieval structure. Torque coupler 106 may exert distally-directed force FD on IMD 110 when a distal force is exerted (e.g., by a clinician) on driver body proximal portion 107. In examples, driver body 104 extends through a delivery catheter lumen 142 of a delivery catheter 134.

The technique includes transferring the torque from torque coupler 106 to IMD 110 using the plurality of protrusions 154 (1606). Torque coupler 106 may transfer the torque when coupler volume 112 receives retrieval structure 111 and coupler axis LC defines the oblique angle with IMD axis LD. Torque coupler 106 may transfer the torque when at least a first protrusion 156 of the plurality of protrusions 154 contacts retrieval structure 111 and the proximally-directed force FP and/or the distally directed force FD generate a contact force providing the engagement force. Torque coupler 106 may cause IMD 110 to rotate about IMD axis LD when torque coupler 106 transfers the torque from torque coupler 106 to IMD 110. In examples, torque coupler 106 causes an attachment member 118 of IMD 110 to disengage from tissue within or in proximity to a target site 120 within the patient when torque coupler 106 causes IMD 110 to rotate. In examples, torque coupler 106 causes attachment member 118 of IMD 110 to engage tissue within or in proximity to target site 120 when torque coupler 106 causes IMD 110 to rotate.

The technique may include slidably translating first protrusion 156 over IMD retrieval structure 111 when the proximally-directed force FP and/or the distally directed force FD generate a contact force providing a slipping force. Torque coupler 106 may rotate relative to IMD retrieval structure 111 around coupler axis LC to cause first protrusion 156 to slidably translate over IMD retrieval structure 111. In examples, proximally-directed force FP and/or distally-directed-force FD may be altered (e.g., by a clinician exerting forces on snare body proximal portion 123 and/or driver body proximal portion 107) to alter the contact force, such that the contact force provides the engagement force and causes first protrusion 156 to transfer the torque to IMD retrieval structure 111. In examples, first protrusion 156 exerts a force F on an IMD petal 162, 164 of IMD retrieval structure 111 to transfer the torque. IMD petal 162, 164 may extend in a direction radially outward from IMD axis LD.

In examples, slidably translating first protrusion 156 over IMD retrieval structure 111 includes slidably translating a rounded corner of first protrusion over IMD retrieval structure 111. In examples, slidably translating first protrusion 156 over IMD retrieval structure 111 includes slidably translating first protrusion 156 over a rounded corner of IMD petal 162, 164. First protrusion 156 may distribute the force F over a protrusion bearing surface 198 extending radially in a direction toward coupler axis LC when first protrusion 156 contacts IMD retrieval structure 111. IMD petal 162, 164 may distribute the force F imparted over an IMD bearing surface 202 defined by at least one of IMD petal 162 or IMD petal 164. In some examples, protrusion 156 transfers force F from protrusion bearing surface 198 to IMD bearing surface 202.

The technique may include positioning, using a delivery catheter 134, at least a portion of driver body 104, torque coupler 106, snare 122, and/or IMD 110 within a delivery receptacle 140 of delivery catheter 134. Delivery catheter 134 may transport at least a portion of driver body 104, torque coupler 106, snare 122, and/or IMD 110 through vasculature of the patient at least one of to retrieve IMD 110 from an anatomical volume of a patient and or implant IMD 110 within the anatomical volume of the patient. In examples, driver body 104 and/or snare body 124 may cause driver body 104, torque coupler 106, snare 122, and/or IMD 110 to position within delivery receptacle volume 168 by slidably translating through a delivery catheter lumen 142 defined by delivery catheter 134.

The disclosure includes the following examples.

Example 1: A medical system, comprising: a driver including a driver body and a torque coupler configured to receive a torque from the driver body, wherein the driver body defines a lumen which opens to a coupler volume defined by the torque coupler, wherein the torque coupler defines a coupler axis extending through the coupler volume and a plurality of protrusions extending toward the coupler axis, wherein the coupler volume is configured to receive a retrieval structure of an implantable medical device when the coupler axis defines an oblique angle with an IMD axis of the implantable medical device, and wherein the torque coupler is configured to exert a distally-directed force on the retrieval structure when the coupler volume receives the retrieval structure; and a snare configured to translate within the lumen, wherein the snare is configured to exert a proximally-directed force on the implantable medical device when the snare translates within the lumen, wherein the snare and the torque coupler are configured to generate an engagement force from the torque coupler to the retrieval structure when at least one of the snare exerts the proximally-directed force or the torque coupler exerts the distally-directed force, wherein the plurality of protrusions are configured to transfer the torque from the torque coupler to the retrieval structure when: the coupler volume receives the retrieval structure, the coupler axis defines the oblique angle with the IMD axis, the snare and the torque coupler generate the engagement force, and at least a first protrusion of the plurality of protrusions contacts the retrieval structure.

Example 2: The medical system of example 1, wherein the snare and the torque coupler are configured to generate the engagement force when the snare exerts the proximally-directed force and the torque coupler exerts the distally-directed force.

Example 3: The medical system of example 1 or example 2, wherein the first protrusion is configured to slidably engage the retrieval structure when: the coupler volume receives the retrieval structure, the torque coupler rotates relative to the retrieval structure, and the snare and the torque coupler generate a slipping force from the torque coupler to the retrieval structure when at least one of the snare exerts the proximally-directed force or the torque coupler exerts the distally-directed force, wherein the slipping force is insufficient to cause the plurality of protrusions to transfer the torque from the torque coupler to the retrieval structure.

Example 4: The medical system of example 3, wherein the snare and the torque coupler are configured to generate the engagement force at a first force magnitude and generate the slipping force at a second force magnitude, wherein the first force magnitude is greater than the second force magnitude.

Example 5: The medical system of example 3 or example 4, wherein the snare and the torque coupler are configured to generate the slipping force when the snare exerts the proximally-directed force and the torque coupler exerts the distally-directed force.

Example 6: The medical system of any of examples 1-5, wherein the torque coupler defines at least three protrusions.

Example 7: The medical system of example 6, wherein the oblique angle is greater than at least one of about 10 degrees, about 20 degrees, or about 30 degrees.

Example 8: The medical system of any of examples 1-7, wherein the torque coupler is configured to exert the distally-directed force in a direction substantially opposite the proximally-directed force exerted by the snare.

Example 9: The medical system of any of examples 1-8, wherein the torque coupler is configured to reduce an angular displacement of the oblique angle when the snare and the torque coupler generate the engagement force.

Example 10: The medical system of any of examples 1-9, wherein the torque coupler includes a coupler surface defining the coupler volume and defining the plurality of protrusions, wherein the plurality of protrusions includes the first protrusion and a second protrusion, and wherein the coupler surface defines a base portion separating the first protrusion and the second protrusion.

Example 11: The medical system of example 10, wherein the first protrusion defines a convexity with respect to the coupler axis.

Example 12: The medical system of example 10 or example 11, wherein the base portion defines a concavity with respect to the coupler axis.

Example 13: The medical system of any of examples 1-12, wherein at least the first protrusion defines one or more rounded corners configured to slidably engage the retrieval structure when the coupler volume receives the retrieval structure and the torque coupler rotates substantially about the coupler axis relative to the retrieval structure.

Example 14: The medical system of example 13, wherein at least one of the rounded corners defines at least one of a curved profile or a curvilinear profile in a plane substantially parallel to the coupler axis.

Example 15: The medical system of example 13 or example 14, wherein at least one of the rounded corners defines at least one of a curved profile or a curvilinear profile in a plane substantially containing the coupler axis.

Example 16: The medical system of any of examples 13-15, wherein at least one of the rounded corners defines at least one of a curved profile or a curvilinear profile in a plane substantially perpendicular to the coupler axis.

Example 17: The medical system of any of examples 1-16, wherein the lumen defines a lumen opening which opens into the coupler volume, and wherein the torque coupler defines a coupler opening that opens into the coupler volume, and wherein at least a portion of the coupler surface radially expands outward from the coupler axis as the coupler surface extends from the lumen opening to the coupler opening.

Example 18: The medical system of example 17, wherein the base portion radially expands outward from the coupler axis as the base portion extends in a direction from the lumen opening to the coupler opening.

Example 19: The medical system of example 17 or example 18, wherein a portion of the coupler surface defining the first protrusion increasingly extends inward toward the coupler axis as the portion of the coupler surface extends in a direction from the lumen opening to the coupler opening.

Example 20: The medical system of any of examples 1-19, wherein the first protrusion defines a protrusion bearing surface extending radially in a direction toward the coupler axis, wherein the first protrusion is configured to distribute a force over the protrusion bearing surface when the first protrusion contacts the retrieval structure and the plurality of protrusions transfers the torque to the retrieval structure.

Example 21: The medical system of any of examples 1-20, wherein a distal end of the torque coupler defines a end coupler perimeter surrounding the coupler opening of example 17, and wherein at least a section of the end coupler perimeter defines a plane, wherein the plane is defines an angle between the plane and the coupler axis.

Example 22: The medical system of example 21, wherein the plane defines an angle of less than 80 degrees between the plane and the coupler axis.

Example 23: The medical system of any of examples 1-22, wherein the snare is configured to exert the proximally-directed force on the retrieval structure.

Example 24: The medical system of any of examples 1-23, wherein the coupler surface at least partially surrounds the coupler axis.

Example 25: The medical system of any of examples 1-24, wherein the torque coupler is configured to rotate substantially about the coupler axis relative to the snare when snare engages the implantable medical device.

Example 26: The medical system of any of examples 1-25, further comprising the implantable medical device.

Example 27: The medical system of example 26, wherein the retrieval structure defines a plurality of petals extending radially outward from the IMD axis.

Example 28: The medical system of example 27, wherein the first protrusion is configured to insert within a recess defined by a first petal of the plurality of petals and a second petal of the plurality of petals when the coupler volume receives the implantable medical device.

Example 29: The medical system of example 27 or example 28, wherein at least one petal defines a petal bearing surface extending radially from the IMD axis, wherein the at least one petal is configured to distribute a force imparted from the first protrusion over the bearing surface when the first protrusion contacts the retrieval structure and the plurality of protrusions transfer the torque from the torque coupler to the retrieval structure.

Example 30: The medical system of any of examples 27-29, wherein at least one petal defines one or more rounded corners configured to slidably engage the at least one of the first protrusion or the second protrusion when the coupler volume receives the implantable medical device and the torque coupler rotates substantially about the coupler axis relative to the retrieval structure.

Example 31: The medical system of example 30, wherein at least one of the rounded corners defines at least one of a curved profile or a curvilinear profile in a plane substantially parallel to the IMD axis.

Example 32: The medical system of example 30 or example 31, wherein at least one of the rounded corners defines at least one of a curved profile or a curvilinear profile in a plane substantially containing the IMD axis.

Example 33: The medical system of any of examples 30-32, wherein at least one of the rounded corners defines at least one of a curved profile or a curvilinear profile in a plane substantially perpendicular to the IMD axis.

Example 34: The medical system of any of examples 30-33, wherein the retrieval structure defines one or more surfaces configured to conform with the coupler surface of example 10 when the coupler volume receives the implantable medical device.

Example 35: A method, comprising: imparting, by a driver body of a driver, a torque on a torque coupler of the driver, wherein the driver body defines a lumen opening to a coupler volume defined by the torque coupler, wherein the torque coupler defines a coupler axis extending through the coupler volume and a plurality of protrusions extending toward the coupler axis, and wherein the coupler volume is configured to receive a retrieval structure of an implantable medical device when the coupler axis defines an oblique angle with an IMD axis of the implantable medical device; imparting at least one of a distally-directed force, exerted by the torque coupler, on the retrieval structure or a proximally-directed force, exerted by a snare, on the IMD to generate an engagement force from the torque coupler to the retrieval structure, wherein the snare is configured to translate within the lumen; and transferring the torque from the torque coupler to the retrieval structure using the plurality of protrusions when the coupler volume receives the retrieval structure, the coupler axis defines the oblique angle with the IMD axis, and at least a first protrusion of the plurality of protrusions contacts the retrieval structure.

Example 36: The method of example 35, further comprising imparting both the proximally-directed force and the distally-directed force.

Example 37: The method of example 35 or example 36, further comprising slidably engaging the first protrusion and the retrieval structure when the coupler volume receives the retrieval structure, the torque coupler rotates relative to the retrieval structure, and the snare and the torque coupler generate a slipping force from the torque coupler to the retrieval structure when at least one of the snare exerts the proximally-directed force or the torque coupler exerts the distally-directed force, wherein the slipping force is insufficient to cause the plurality of protrusions to transfer the torque from the torque coupler to the retrieval structure.

Example 38: The method of any of examples 35-37, further comprising generating the slipping force when the snare exerts the proximally-directed force and the torque coupler exerts the distally-directed force.

Example 39: The method of any of examples 35-38, medical system of example 6, further comprising receiving the retrieval structure in the coupler volume when the oblique angle is greater than at least one of about 10 degrees, about 20 degrees, or about 30 degrees.

Example 40: The method of any of examples 35-39, further comprising exerting the distally-directed force in a direction substantially opposite the proximally-directed force.

Example 41: The method of any of examples 35-40, further comprising reducing, using at least one of the proximally-directed force or the distally-directed force, an angular displacement of the oblique angle.

Example 42: The method of any of examples 35-41, further comprising slidably engaging one or more rounded corners of the first protrusion and the retrieval structure.

Example 43: The method of any of examples 35-42, further comprising distributing a force, by the first protrusion, over a protrusion bearing surface extending radially in a direction toward the coupler axis when the first protrusion contacts the retrieval structure and the plurality of protrusions transfers the torque to the retrieval structure.

Example 44: The method of any of examples 35-43, further comprising exerting the proximally-directed force on the retrieval structure.

Example 45: The method of any of examples 35-44, further comprising rotating the torque coupler about the coupler axis relative to the snare when snare engages the implantable medical device.

Example 46: The method of any of examples 35-45, further comprising rotating the torque coupler about the coupler axis relative to the retrieval structure when torque coupler exerts the distally-directed force.

Example 47: The method of any of examples 35-46, further comprising rotating the torque coupler about the coupler axis relative to the retrieval structure to cause the first petal to insert between a first petal defined by the retrieval structure and a second petal defined by the retrieval structure.

Example 48: The method of any of examples 35-47, further comprising distributing a force imparted from the first petal over an IMD bearing surface defined by at least one IMD petal, wherein the petal bearing surface extend in a radial direction away from the IMD axis.

Example 49: The method of any of examples 35-48, further comprising slidably engaging one or more rounded corners of at least one IMD petal with the first protrusion.

Various examples of the disclosure have been described. Any combination of the described systems, operations, or functions is contemplated. These and other examples are within the scope of the following claims.

1: A medical system, comprising:
a driver including a driver body and a torque coupler configured to receive a torque from the driver body,
wherein the driver body defines a lumen which opens to a coupler volume defined by the torque coupler,
wherein the torque coupler defines a coupler axis extending through the coupler volume and a plurality of protrusions extending toward the coupler axis,
wherein the coupler volume is configured to receive a retrieval structure of an implantable medical device when the coupler axis defines an oblique angle with an IMD axis of the implantable medical device, and
wherein the torque coupler is configured to exert a distally-directed force on the retrieval structure when the coupler volume receives the retrieval structure; and
a snare configured to translate within the lumen,
wherein the snare is configured to exert a proximally-directed force on the implantable medical device when the snare translates within the lumen,
wherein the snare and the torque coupler are configured to generate an engagement force from the torque coupler to the retrieval structure when at least one of the snare exerts the proximally-directed force or the torque coupler exerts the distally-directed force,
wherein the plurality of protrusions are configured to transfer the torque from the torque coupler to the retrieval structure when:
the coupler volume receives the retrieval structure,
the coupler axis defines the oblique angle with the IMD axis,
the snare and the torque coupler generate the engagement force, and
at least a first protrusion of the plurality of protrusions contacts the retrieval structure.

2: The medical system of claim 1, wherein the first protrusion is configured to slidably engage the retrieval structure when:
the coupler volume receives the retrieval structure,
the torque coupler rotates relative to the retrieval structure, and
the snare and the torque coupler generate a slipping force from the torque coupler to the retrieval structure when at least one of the snare exerts the proximally-directed force or the torque coupler exerts the distally-directed force,
wherein the slipping force is insufficient to cause the plurality of protrusions to transfer the torque from the torque coupler to the retrieval structure.

4-15. (canceled)

16: The medical system of claim 1, wherein the oblique angle is greater than at least one of about 10 degrees, about 20 degrees, or about 30 degrees.

17: The medical system of claim 1, wherein the torque coupler is configured to reduce an angular displacement of the oblique angle when the snare and the torque coupler generate the engagement force.

18: The medical system of claim 1, wherein the torque coupler includes a coupler surface defining the coupler volume and defining the plurality of protrusions, wherein the plurality of protrusions includes the first protrusion and a second protrusion, and wherein the coupler surface defines a base portion separating the first protrusion and the second protrusion.

19: The medical system of claim 18, wherein the first protrusion defines a convexity with respect to the coupler axis.

20: The medical system of claim 18, wherein the base portion defines a concavity with respect to the coupler axis.

21: The medical system of claim 1, wherein at least the first protrusion defines one or more rounded corners configured to slidably engage the retrieval structure when the coupler volume receives the retrieval structure and the torque coupler rotates substantially about the coupler axis relative to the retrieval structure.

22: The medical system of claim 1, wherein the lumen defines a lumen opening which opens into the coupler volume, and wherein the torque coupler defines a coupler opening that opens into the coupler volume, and wherein at least a portion of the coupler surface radially expands outward from the coupler axis as the coupler surface extends from the lumen opening to the coupler opening.

23: The medical system of claim 22, wherein a portion of the coupler surface defining the first protrusion increasingly extends inward toward the coupler axis as the portion of the coupler surface extends in a direction from the lumen opening to the coupler opening.

24: The medical system of claim 1, wherein the first protrusion defines a protrusion bearing surface extending radially in a direction toward the coupler axis, wherein the first protrusion is configured to distribute a force over the protrusion bearing surface when the first protrusion contacts the retrieval structure and the plurality of protrusions transfers the torque to the retrieval structure.

25: The medical system of claim 1, wherein a distal end of the torque coupler defines an end coupler perimeter, wherein at least a section of the end coupler perimeter defines a plane, wherein the plane is defines an angle between the plane and the coupler axis, and wherein the plane defines an angle of less than 80 degrees between the plane and the coupler axis.

26: The medical system of claim 1, further comprising the implantable medical device, wherein the retrieval structure defines a plurality of petals extending radially outward from the IMD axis, and wherein the first protrusion is configured to insert within a recess defined by a first petal of the plurality of petals and a second petal of the plurality of petals when the coupler volume receives the implantable medical device.

27: The medical system of claim 26, wherein at least one petal defines one or more rounded corners configured to slidably engage the first protrusion when the coupler volume receives the implantable medical device and the torque coupler rotates substantially about the coupler axis relative to the retrieval structure.

28: The medical system of claim 1, wherein the torque coupler is configured to exert the distally-directed force in a direction substantially opposite the proximally-directed force exerted by the snare.

29: A medical system, comprising:
a driver including a driver body and a torque coupler configured to receive a torque from the driver body, wherein the driver body defines a lumen which opens to a coupler volume defined by a coupler surface of the torque coupler, wherein the torque coupler defines a coupler axis extending through the coupler volume and the lumen, wherein the coupler volume is configured to receive a retrieval structure of an implantable medical device when the coupler axis defines an oblique angle with an IMD axis of the implantable medical device, and wherein:
the coupler surface defines a first protrusion extending toward the coupler axis, a second protrusion extending toward the coupler axis, and a base portion separating the first protrusion and the second protrusion,
at least the first protrusion defines a convexity with respect to the coupler axis,
at least the first protrusion defines one or more rounded corners configured to slidably engage the retrieval structure when the coupler volume receives the retrieval structure and the torque coupler rotates substantially about the coupler axis relative to the retrieval structure, and
the base portion defines a concavity with respect to the coupler axis; and
a snare configured to translate within the lumen,
wherein the snare is configured to exert a proximally-directed force on the implantable medical device when the snare translates within the lumen, and
wherein the torque coupler is configured to exert a distally-directed force on the retrieval structure when the coupler volume receives the retrieval structure and the snare is configured to exert a proximally-directed force.

30: The medical system of claim 29, wherein at least one of the one or more rounded corners defines at least one of a curved profile or a curvilinear profile in a plane substantially parallel to the coupler axis or in a plane substantially containing the coupler axis.

31: The medical system of claim 29, wherein the lumen defines a lumen opening which opens into the coupler volume, and wherein the torque coupler defines a coupler opening that opens into the coupler volume, and wherein at least a portion of the coupler surface radially expands outward from the coupler axis as the coupler surface extends from the lumen opening to the coupler opening.

32: A method, comprising:
imparting, by a driver body of a driver, a torque on a torque coupler of the driver, wherein the driver body defines a lumen opening to a coupler volume defined by the torque coupler, wherein the torque coupler defines a coupler axis extending through the coupler volume and a plurality of protrusions extending toward the coupler axis, and wherein the coupler volume is configured to receive a retrieval structure of an implantable medical device when the coupler axis defines an oblique angle with an IMD axis of the implantable medical device;
imparting at least one of a distally-directed force, exerted by the torque coupler, on the retrieval structure or a proximally-directed force, exerted by a snare, on the IMD to generate an engagement force from the torque coupler to the retrieval structure, wherein the snare is configured to translate within the lumen; and
transferring the torque from the torque coupler to the retrieval structure using the plurality of protrusions when the coupler volume receives the retrieval structure, the coupler axis defines the oblique angle with the IMD axis, and at least a first protrusion of the plurality of protrusions contacts the retrieval structure.

33: The method of claim 32, further comprising imparting both the proximally-directed force and the distally-directed force.
